(19) United States
(12) Patent Application Publication
(10) Pub. No.: US 2019/0084909 A1
(43) Pub. Date: Mar. 21, 2019

(54) FLUORINATED CBD COMPOUNDS, COMPOSITIONS AND USES THEREOF

(71) Applicants: YISSUM RESEARCH DEVELOPMENT COMPANY OF THE HEBREW UNIVERSITY OF JERUSALEM, Jerusalem (IL); UNIVERSIDADE FEDERAL DO RIO GRANDE DO SUL-UFRGS, Porto Alegre (BR); UNIVERSIDADE DE SÃO PAULO - USP, São Paulo (BR)

(72) Inventors: Raphael Mechoulam, Jerusalem (IL); Flavio Pereira Kapczinski, Porto Alegre (BR); Jose Alexandre De Souza Crippa, Ribeirao Preto (BR); Antonio Waldo Zuardi, Ribeirao Preto (BR); Jaime Eduardo Cecilio Hallak, Ribeirao Preto (BR); Francisco Silveira Guimaraes, Ribeirao Preto (BR); Aviva Breuer, Jerusalem (IL)

(21) Appl. No.: 15/745,306

(22) PCT Filed: Jul. 15, 2016
(86) PCT No.: PCT/BR16/50162

§ 371 Date: Jan. 16, 2018

Related U.S. Application Data
(60) Provisional application No. 62/193,296, filed on Jul. 16, 2015.
(60) Provisional application No. 62/255,738, filed on Nov. 16, 2015.

(51) Int. Cl. C07C 39/42; A61P 25/16; A61P 25/28; A61P 25/24; A61P 25/18; A61P 25/08; A61P 25/22; C07C 69/63

(52) U.S. Cl. CPC C07C 39/42; A61P 25/16; A61P 25/28; A61P 25/24; C07C 2601/16; A61P 25/08; A61P 25/22; C07C 69/63; A61P 25/18

(57) ABSTRACT

The present invention relates to fluorine substituted CBD compounds, compositions thereof and uses thereof for the preparation of medicaments.

### CROSS-REFERENCE TO RELATED APPLICATIONS

This application claims the benefit of priority to U.S. Provisional Application No. 62/193,296, filed on Jul. 16, 2015, and 62/255,738 filed on Nov. 16, 2015, entitled “FLUORINATED CBD COMPOUNDS, COMPOSITIONS AND USES THEREOF”, the disclosures of which are hereby incorporated by reference in their entireties for all purposes.

### TECHNOLOGICAL FIELD

The present invention relates to fluorine substituted CBD compounds, compositions thereof and uses thereof for the preparation of medicaments.

### GENERAL DESCRIPTION

In one aspect of the present invention provides a compound having the general formula (I):

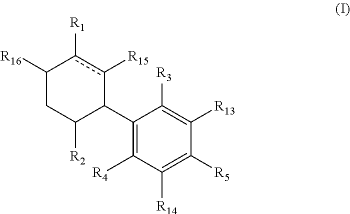

wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R2 is selected from straight or branched C1-C8 alkyl, straight or branched C2-C10 alkenyl, straight or branched C2-C10 alkynyl, each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is selected from a straight or branched C5-C12 alkyl, a straight or branched C5-C9 alkoxy, a straight or branched C1-C7 ether, each being optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13, R14, R15 and R16 are each optionally selected from H and F; provided that at least one of R13, R14, R15 and R16 is F or at least one of R1 and R2 is substituted with F.

In one embodiment, compound having the general formula (I) excludes:

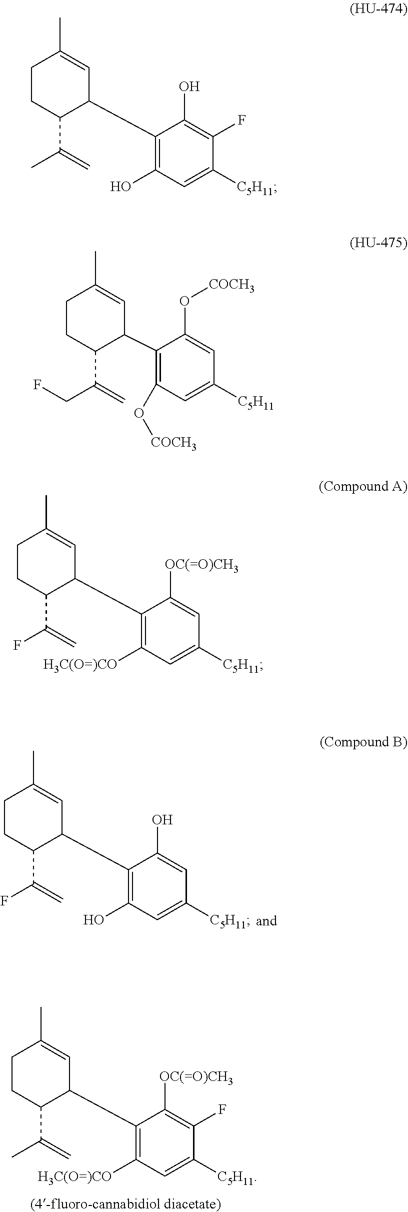

In another aspect of the present invention provides a compound having the general formula (Ia):

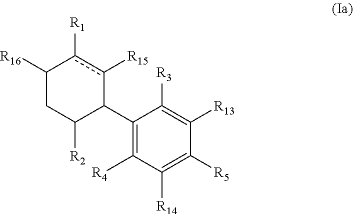

wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R2 is selected from straight or branched C1-C8 alkyl, straight or branched C2-C10 alkenyl, straight or branched C2-C10 alkynyl, each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is a straight or branched C5 alkyl, optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13, R14, R15 and R16 are each optionally selected from H and F; provided that at least one of R13, R14, R15 and R16 is F or at least one of R1 and R2 is substituted with F.

In one embodiment, compound having the general formula (Ia) excludes: HU-474, HU-475, Compound A, Compound B, and 4′-fluoro-cannabidiol diacetate.

In another one of its aspects the invention provides a compound having the general formula (II):

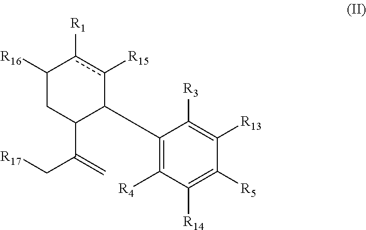

wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is selected from a straight or branched C5-C12 alkyl, a straight or branched C5-C9 alkoxy, a straight or branched C1-C7 ether, each being optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13, R14, R15, R16 and R17 are each optionally selected from H and F; provided that at least one of R13, R14, R15 and R16 is F or R1 is substituted with F.

In one embodiment, compound having the general formula (II) excludes HU-474 and 4′-fluoro-cannabidiol diacetate.

In another one of its aspects the invention provides a compound having the general formula (IIa):

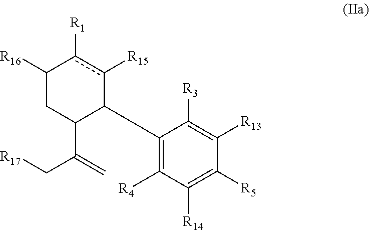

wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is a straight or branched C5 alkyl, optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13, R14, R15, R16 and R17 are each optionally selected from H and F; provided that at least one of R13, R14, R15 and R16 is F or R1 is substituted with F.

In one embodiment, compound having the general formula (IIa) excludes HU-474 and 4′-fluoro-cannabidiol diacetate.

In another one of its aspects the invention provides a compound having the general formula (III):

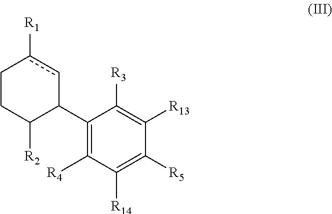

wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9;
R2 is selected from straight or branched C1-C8 alkyl, straight or branched C2-C10 alkenyl, straight or branched C2-C10 alkynyl;
R3 and R4 are each independently selected from H, straight or branched C1-C5 alkyl, —OR10, —C(═O)R11, —OC(═O)R12; provided that at least one of R3 and R4 is different than H;
R5 is selected from a straight or branched C5-C12 alkyl, a straight or branched C5-C9 alkoxy, a straight or branched C1-C7 ether, each being optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13 and R14 are each optionally selected from H and F;
provided that at least one of R13 and R14 are F.

In one embodiment, compound having the general formula (III) excludes HU-474 and 4′-fluoro-cannabidiol diacetate.

In another one of its aspects the invention provides a compound having the general formula (IIIa):

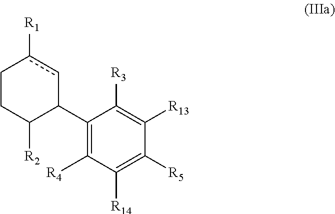

wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9;
R2 is selected from straight or branched C1-C8 alkyl, straight or branched C2-C10 alkenyl, straight or branched C2-C10 alkynyl;
R3 and R4 are each independently selected from H, straight or branched C1-C5 alkyl, —OR10, —C(═O)R11, —OC(═O)R12; provided that at least one of R3 and R4 is different than H;
R5 is a straight or branched C5 alkyl, optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13 and R14 are each optionally selected from H and F;
provided that at least one of R13 and R14 are F.

In one embodiment, compound having the general formula (IIIa) excludes HU-474 and 4′-fluoro-cannabidiol diacetate.

In another one of its aspects the invention provides a compound of general formula (IV)

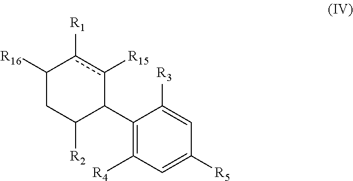

wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R2 is selected from straight or branched C1-C8 alkyl, straight or branched C2-C10 alkenyl, straight or branched C2-C10 alkynyl, each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is selected from a straight or branched C5-C12 alkyl, a straight or branched C5-C9 alkoxy, a straight or branched C1-C7 ether, each being optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R15 and R16 are each optionally selected from H and F;
provided that at least one of R15 and R16 is F or at least one of R1 and R2 is substituted with F.

In one embodiment, compound having the general formula (IV) excludes HU-475, Compound A, and Compound B.

In another one of its aspects the invention provides a compound of general formula (IVa)

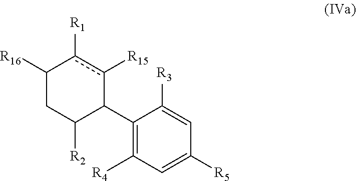

wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R2 is selected from straight or branched C1-C8 alkyl, straight or branched C2-C10 alkenyl, straight or branched C2-C10 alkynyl, each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is a straight or branched C5 alkyl, optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R15 and R16 are each optionally selected from H and F;
provided that at least one of R15 and R16 is F or at least one of R1 and R2 is substituted with F.

In one embodiment, compound having the general formula (IVa) excludes HU-475, Compound A, and Compound B.

In some embodiments [symbol] is a double bond.

In some other embodiments R1 straight or branched C1-C8 alkyl; R3 and R4 are each independently —OR10; R10 is selected from H, a straight or branched C1-C5 alkyl.

In further embodiments R1 is straight or branched C1-C8 alkyl, and R3 and R4 are OH.

In other embodiments, R3 and R4 are each independently selected from H, —OR10, and —OC(═O)R12; R10 is selected from H, a straight or branched C1-C5 alkyl; and R12 is selected from H, OH, straight or branched C1-C5 alkyl, —NH3, straight or branched C1-C5 amine.

In some embodiments, R5 is a straight or branched C5-C12 alkyl.

In some embodiments, R5 is a straight or branched C5 alkyl.

In further embodiments of a compound of the invention at least one of R13, R14, R15 and R16 is F.

In other embodiments of a compound of the invention at least one of R13 and R14 is F.

In further embodiments of a compound of the invention at least one of R15 and R16 is F.

In other embodiments of a compound of the invention at least one of R1 and R2 is substituted with F.

In yet further embodiments of a compound of the invention R1 is selected from straight or branched C1-C8 alkyl, straight or branched C2-C10 alkenyl, straight or branched C2-C10 alkynyl, each being substituted by F.

In further embodiments of a compound of the invention R2 is selected from straight or branched C1-C8 alkyl, straight or branched C2-C10 alkenyl, straight or branched C2-C10 alkynyl, each substituted by F.

In some embodiments, a compound of formula (I), (Ia), (II), (IIa), (III) and (IIIa) excludes HU-474 and 4′-fluoro-cannabidiol diacetate.

In some embodiments, a compound of formula (I), (la), (IV) and (IVa) excludes HU-475, Compound A, and Compound B.

In further embodiments a compound of the invention is of the general formula (V):

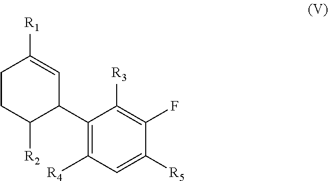

wherein R1, R2, R3, R4 and R5 are as defined therein.

In one embodiment, R5 is a straight or branched C5-C12 alkyl in formula (V).

In another embodiment, R5 is a straight or branched C5 alkyl in formula (V).

In some embodiments, compound having the general formula (V) excludes: HU-474 and 4′-fluoro-cannabidiol diacetate.

In other embodiments a compound of the invention is of the general formula (VI):

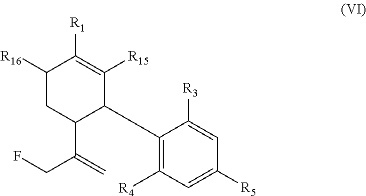

wherein R1, R3, R4, R5, R15 and R16 are as defined therein.

In one embodiment, R5 is a straight or branched C5-C12 alkyl in formula (VI).

In another embodiment, R5 is a straight or branched C5 alkyl in formula (VI).

In some embodiments, a compound of formula (VI) excludes HU-475.

The present invention provides a compound having the general formula (I):

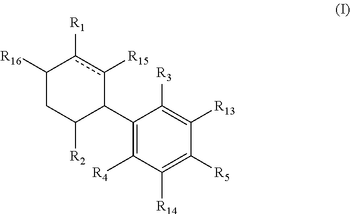

wherein [symbol]
is a double bond;
R1 is a straight or branched C1-C8 alkyl optionally substituted by at least one F;
R2 is a straight or branched C2-C10 alkenyl optionally substituted by at least one F;
R3 and R4 are each independently selected from H, —OR10, —OC(═O)R12; provided that at least one of R3 and R4 is different than H;
R5 is a straight or branched C5-C12 alkyl optionally substituted by at least one substituent selected from —OH, —NH3, straight or branched C1-C5 amine, halogen, phenyl, aryl, heteroaryl, cycloalkyl and heterocycloalkyl;
R10 is selected from H, a straight or branched C1-C5 alkyl; and
R12 is selected from H, OH, straight or branched C1-C5 alkyl, straight or branched C1-C5 alkoxy, —NH3, straight or branched C1-C5 amine;
R13, R14, R15 and R16 are each optionally selected from H and F; provided that at least one of R13, R14, R15 and R16 is F or at least one of R1 and R2 is substituted with F.

The present invention also provides a compound having the general formula (Ia):

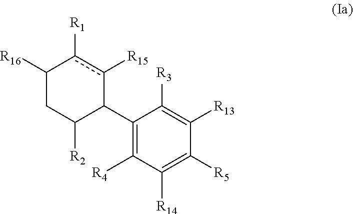

wherein [symbol]
is a double bond;
R1 is a straight or branched C1-C8 alkyl optionally substituted by at least one F;
R2 is a straight or branched C2-C10 alkenyl optionally substituted by at least one F;
R3 and R4 are each independently selected from H, —OR10, —OC(═O)R12; provided that at least one of R3 and R4 is different than H;
R5 is a straight or branched C5 alkyl optionally substituted by at least one substituent selected from —OH, —NH3, straight or branched C1-C5 amine, halogen, phenyl, aryl, heteroaryl, cycloalkyl and heterocycloalkyl;
R10 is selected from H, a straight or branched C1-C5 alkyl; and
R12 is selected from H, OH, straight or branched C1-C5 alkyl, straight or branched C1-C5 alkoxy, —NH3, straight or branched C1-C5 amine;
R13, R14, R15 and R16 are each optionally selected from H and F; provided that at least one of R13, R14, R15 and R16 is F or at least one of R1 and R2 is substituted with F.

The invention further provides a compound having the formula:

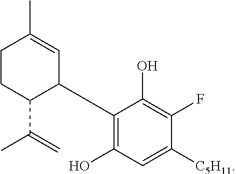

The invention further provides a compound having the formula:

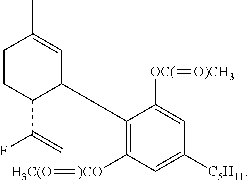

The invention further provides a compound having the formula:

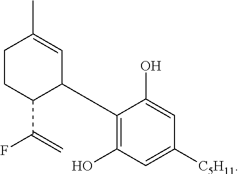

The invention further provides a compound having the formula:

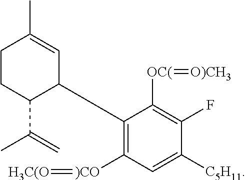

The invention further provides a compound having the formula:

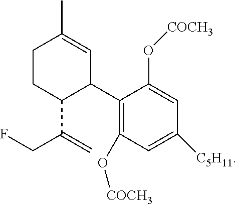

The invention further provides a compound having the formula:

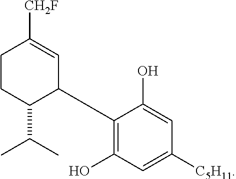

The invention further provides a compound having the formula:

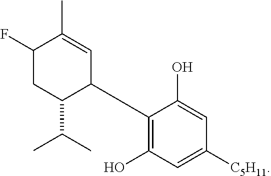

The invention further provides a compound having the formula:

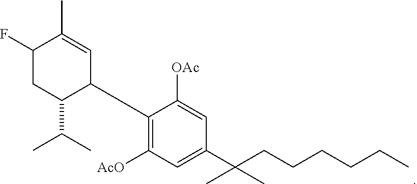 [SMILES: CCCCCCC(C)(C)c1cc(OC(C)=O)c(C2C=C(C)C(F)C[C@H]2C(C)C)c(OC(C)=O)c1 ; "."]

The invention further provides a compound having the formula:

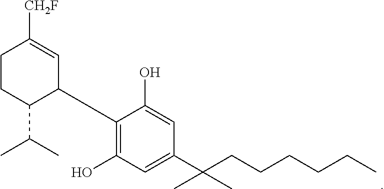

The invention further provides a compound having the formula:

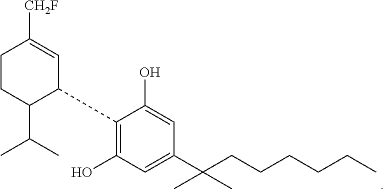

The invention further provides a compound having the formula:

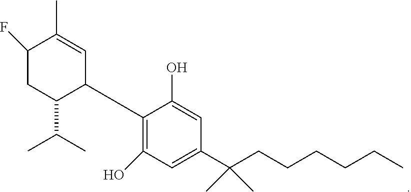 [SMILES: CCCCCCC(C)(C)c1cc(O)c(C2C=C(C)C(F)C[C@H]2C(C)C)c(O)c1 ; "."]

The term “straight or branched C1-C8 alkyl” should be understood to encompass a straight or branched hydrocarbon chain having 1, 2, 3, 4, 5, 6, 7 or 8 carbon atoms, wherein all bonds are single bonds.

The term “straight or branched C2-C10 alkenyl” should be understood to encompass a straight or branched hydrocarbon chain having 2, 3, 4, 5, 6, 7, 8, 9 or 10 carbon atoms, having at least one double unsaturated bond between at least two carbon atoms.

The term “straight or branched C2-C10 alkynyl” should be understood to encompass a straight or branched hydrocarbon chain having 2, 3, 4, 5, 6, 7, 8, 9 or 10 carbon atoms, having at least one triple unsaturated bond between at least two carbon atoms.

The term “each optionally substituted by at least one F” should be understood to relate to the option of having at least one fluor atom substituted on any of the substituents such as R1 and/or R2 at any position, replacing at least one hydrogen atom.

The term “straight or branched C5-C9 alkoxy” should be understood to encompass a radical of —OR wherein R is a straight or branched alkyl having 5, 6, 7, 8 or 9 carbon atoms.

The term “straight or branched C1-C7 ether” should be understood t encompass a radical of —R′OR wherein R is a straight or branched alkyl having 1, 2, 3, 4, 5, 6 or 7, carbon atoms and R′ is a straight or branched alkanyl having 1, 2, 3, 4, 5, 6 or 7, carbon atoms.

The term “straight or branched C1-C5 amine” should be understood to encompass a primary (—NH2R), secondary (—NHRR′) or tertiary amine (—N+RR′R″) wherein R, R′ and R″ are each independently a straight or branched alkyl having 1, 2, 3, 4 or 5 carbon atoms.

The term “halogen” should be understood to encompass any halogen atoms including F, Cl, Br and I.

The term “aryl” is meant to encompass an aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

The term “heteroaryl” refers to a monocyclic or multicyclic aromatic ring system, in certain embodiments, of about 5 to about 15 members wherein one or more, in some embodiments between 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl,

The term “cycloalkyl” refers to a monocyclic or multicyclic non-aromatic ring system, in one embodiment of 3 to 10 members, in another embodiment of 4 to 7 members, in further embodiments between 5 to 6 member carbon atoms.

The term “heterocycloalkyl” refers to a monocyclic or multicyclic non-aromatic ring system, in one embodiment of 3 to 10 members, in another embodiment of 4 to 7 members, in a further embodiments between 5 to 6 members, wherein one or more, in certain embodiments between 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidine, or the nitrogen may be quaternary ammonium group where the substituents are selected as above.

When referring to a compound of the invention wherein at least one of R13, R14, R15 and R16 is F or at least one of R1 and R2 is substituted with F, it should be understood to encompass a compound of the invention wherein either at least one of R13, R14, R15 and R16 is F or at least one of R1 and R2 is a substituent as defined herein above wherein at least one of its hydrogen atoms (at any location on the moiety) is substituted by an F atom.

In some other embodiments at least one of R13, R14, R15 and R16 is F and at least one of R1 and R2 is substituted with F. Under this embodiment the invention encompasses a compound wherein at least one of R13, R14, R15 and R16 is F and at least one of R1 and R2 is a substituent as defined herein above wherein at least one of its hydrogen atoms (at any location on the moiety) is substituted by an F atom.

In another one of its aspects the invention provides a composition comprising at least one compound of the invention, as described herein above in all aspects and embodiments of a compound of the invention.

In some embodiments of a composition of the invention, said composition is a pharmaceutical composition.

A pharmaceutical composition of the present invention have potent antioxidant and/or free radical scavenging properties, that prevent or reduce oxidative damage in biological systems, such as occurs in ischemic/reperfusion injury, or in chronic neurodegenerative diseases such as Alzheimer's disease, HIV dementia, and many other oxidation associated diseases.

Thus, the invention provides a composition comprising a compound of the invention (as defined in any aspect and embodiment of a compound of the invention) being an antioxidant composition.

As used herein, an “antioxidant” is a substance that, when present in a mixture containing an oxidizable substrate biological molecule, significantly delays or prevents oxidation of the substrate biological molecule. Antioxidants can act by scavenging biologically important reactive free radicals or other reactive oxygen species (O2−, H2O2, .OH, HOCl, ferryl, peroxyl, peroxynitrite, and alkoxyl), or by preventing their formation, or by catalytically converting the free radical or other reactive oxygen species to a less reactive species.

Relative antioxidant activity can be measured by cyclic voltametry studies, where the voltage (x-axis) is an index of relative antioxidant activity. The voltage at which the first peak occurs is an indication of the voltage at which an electron is donated, which in turn is an index of antioxidant activity.

“Therapeutically effective antioxidant doses” can be determined by various methods, including generating an empirical dose-response curve, predicting potency and efficacy of a congener by using quantitative structure activity relationships (QSAR) methods or molecular modeling, and other methods used in the pharmaceutical sciences. Since oxidative damage is generally cumulative, there is no minimum threshold level (or dose) with respect to efficacy. However, minimum doses for producing a detectable therapeutic or prophylactic effect for particular disease states can be established.

The present invention also relates to pharmaceutical compositions comprising at least one compound of the subject invention in admixture with pharmaceutically acceptable auxiliaries, and optionally other therapeutic agents. The auxiliaries are “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, flavouring agents, anti-oxidants, and wetting agents.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

In another one of its aspects that invention provides a compound of the invention, as described herein above in all aspects and embodiments of a compound of the invention, for use in the treatment of at least one condition, disease or disorder selected from the group consisting of:
psychiatric disorders (none limiting examples include: anxiety and stress, depression, schizophrenia, panic, withdrawal symptoms in cannabis and tobacco addiction, reward-facilitating effect of morphine and cocaine, lowers cannabis and THC effects such as memory loss, psychotic-like symptoms);
inflammation (none limiting examples include: Crohn's disease, inflammatory bowel disease, colitis, pancreatitis, asthma, chronic inflammatory and neuropathic pain);
oxidative associated diseases, conditions or disorders (pathological conditions that result at least in part from the production of or exposure to free radicals, particularly oxyradicals, or reactive oxygen species. It is evident to those of skill in the art that most pathological conditions are multi-factorial, and that assigning or identifying the predominant causal factors for any particular condition is frequently difficult. For these reasons, the term “free radical associated disease” encompasses pathological states that are recognized as conditions in which free radicals or reactive oxygen species (ROS) contribute to the pathology of the disease, or wherein administration of a free radical inhibitor (e.g. desferroxamine), scavenger (e.g. tocopherol, glutathione) or catalyst (e.g. superoxide dismutase, catalase) is shown to produce detectable benefit by decreasing symptoms, increasing survival, or providing other detectable clinical benefits in treating or preventing the pathological state. Oxidative associated diseases include, without limitation, free radical associated diseases, such as ischemia, ischemic reperfusion injury, inflammatory diseases, systemic lupus erythematosis, myocardial ischemia or infarction, cerebrovascular accidents (such as a thromboembolic or hemorrhagic stroke) that can lead to ischemia or an infarct in the brain, operative ischemia, traumatic hemorrhage (for example a hypovolemic stroke that can lead to CNS hypoxia or anoxia), spinal cord trauma, Down's syndrome, Crohn's disease, autoimmune diseases (e.g. rheumatoid arthritis or diabetes), cataract formation, uveitis, emphysema, gastric ulcers, oxygen toxicity, neoplasia, undesired cellular apoptosis, radiation sickness, and others. The present invention is further directed to a compound or composition of the invention used in the treatment of oxidative associated diseases of the CNS. In some embodiments, the pharmaceutical composition of the present invention is used for preventing, arresting, or treating neurological damage in Parkinson's disease, Alzheimer's disease and HIV dementia; autoimmune neurodegeneration of the type that can occur in encephalitis, and hypoxic or anoxic neuronal damage that can result from apnea, respiratory arrest or cardiac arrest, and anoxia caused by drowning, brain surgery or trauma (such as concussion or spinal cord shock)).
rheumatoid arthritis;
cardiovascular diseases (none limiting examples include: reduces infarct size and increase blood flow in stroke; reduces vasoconstriction; lowers vascular damage caused by a high glucose environment; reduces the vascular hyperpermeability);
obesity (none limiting examples include: food consumption; lowering appetite); metabolic syndrome);
diabetes and associated disorders and symptoms (none limiting examples include: type 1 and type 2, cardiomyopathy and retinopathy associated with diabetes);
emesis and nausea;
ischemic/reperfusion injury associated with myocardial;
liver or renal diseases;
hypoxia/ischemia injury;
neuronal damage due to neurological diseases or injury (none limiting examples include: Parkinson's disease; Huntington's disease; Alzheimer's disease; cerebral infarction; hepatic encephalopathy; traumatic brain injury; cerebral ischemia; spinal cord injury; memory rescuing effects); cancer and resistance to cancer chemotherapy (none limiting examples include: cancer cell migration (metastasis); inhibits angiogenesis); epilepsy and convulsions;
   and any condition or symptom associated therefrom.

In further embodiments, said condition, disease, disorder or symptom associated with inflammation is selected from rheumatoid arthritis, multiple sclerosis, inflammatory bowel disease, diabetes and any combinations thereof.

In yet other embodiments, said disease is a psychiatric disease condition or disorder or any symptom associated therewith.

In other embodiments, said psychiatric disease condition or disorder or any symptom associated therewith is selected from anxiety, stress, depression, schizophrenia, panic, substance abuse withdrawal symptoms, reward-facilitating effect of addictive substances, memory loss, psychotic-like symptoms associated with the use of substance abuse.

In another one of its aspects the invention provides a compound of the invention, as described herein above in all aspects and embodiments of a compound of the invention, for use in reduction of oxidative stress.

When referring to “reduction of oxidative stress” it should be understood to encompass any qualitative or quantitative reduction in the oxidative stress in a body tissue or cell of a subject treated with a compound or composition of the invention. Oxidative stress is characterized by an imbalance between the systemic manifestation of reactive oxygen species and a biological system's ability to readily detoxify the reactive intermediates or to repair the resulting damage. Disturbances in the normal redox state of cells can cause toxic effects through the production of peroxides and free radicals that damage all components of the cell, including proteins, lipids, and DNA. Further, some reactive oxidative species act as cellular messengers in redox signaling. Thus, oxidative stress can cause disruptions in normal mechanisms of cellular signaling.

Non limiting list of diseases, conditions or disorders associated with oxidative stress in a cell or tissue of a subject include: cancer, Parkinson's disease, Alzheimer's disease, atherosclerosis, heart failure, myocardial infarction, Schizophrenia, Bipolar disorder, fragile X syndrome, Sickle Cell Disease, lichen planus, vitiligo, autism, and chronic fatigue syndrome.

In a further aspect the invention provides a compound of the invention, as described herein above in all aspects and embodiments of a compound of the invention, for use in the treatment of any disease, condition or disorder caused by or associated with oxidative stress.

Oxidative associated diseases include, without limitation, free radical associated diseases, such as ischemia, ischemic reperfusion injury, inflammatory diseases, systemic lupus erythematosis, myocardial ischemia or infarction, cerebrovascular accidents (such as a thromboembolic or hemorrhagic stroke) that can lead to ischemia or an infarct in the brain, operative ischemia, traumatic hemorrhage (for example a hypovolemic stroke that can lead to CNS hypoxia or anoxia), spinal cord trauma, Down's syndrome, Crohn's disease, autoimmune diseases (e.g. rheumatoid arthritis or diabetes), cataract formation, uveitis, emphysema, gastric ulcers, oxygen toxicity, neoplasia, undesired cellular apoptosis, radiation sickness, and others. The present invention is believed to be particularly beneficial in the treatment of oxidative associated diseases of the CNS, because of the ability of the cannabinoids to cross the blood brain barrier and exert their antioxidant effects in the brain. In some embodiments, the pharmaceutical composition or compound of the present invention is used for preventing, arresting, or treating neurological damage in Parkinson's disease, Alzheimer's disease and HIV dementia; autoimmune neurodegeneration of the type that can occur in encephalitis, and hypoxic or anoxic neuronal damage that can result from apnea, respiratory arrest or cardiac arrest, and anoxia caused by drowning, brain surgery or trauma (such as concussion or spinal cord shock).

In some embodiments, said disease, condition or disorder caused or associated with oxidative stress are selected from the group consisting of cancer, oxidative neurological disorders, free radical associated diseases, ischemia, ischemic reperfusion injury, inflammatory diseases, systemic lupus erythematosis, myocardial ischemia or infarction, cerebrovascular accidents, operative ischemia, traumatic hemorrhage, spinal cord trauma, Down's syndrome, Crohn's disease, autoimmune diseases, cataract formation, uveitis, emphysema, gastric ulcers, oxygen toxicity, neoplasia, undesired cellular apoptosis, radiation sickness, and any combinations thereof.

In a further aspect the invention provides a compound as defined in all aspects and embodiments of a compound of the invention, for use in the treatment of oxidative associated diseases, disorder or condition of the CNS. In another aspect the invention provides a compound as defined in all aspects and embodiments of a compound of the invention, for use in preventing, arresting, or treating neurological damage in a subject suffering from at least one disease, disorder or condition selected from Parkinson's disease, Alzheimer's disease and HIV dementia; autoimmune neurodegeneration, hypoxic or anoxic neuronal damage, respiratory arrest or cardiac arrest, anoxia caused by drowning and brain surgery or trauma.

In a further aspect the invention provides a compound as defined in all aspects and embodiments of a compound of the invention, for use in the treatment of an ischemic or neurodegenerative disease in the central nervous.

In some embodiments of a compound for use above, said ischemic or neurodegenerative disease is selected from the group consisting of: an ischemic infarct, Alzheimer's disease, Parkinson's disease, and human immunodeficiency virus dementia, Down's syndrome, and heart disease or any combinations thereof.

In a further aspect the invention encompasses a use of a compound of the invention, as described herein above in all aspects and embodiments of a compound of the invention, for the manufacture of a medicament (or a pharmaceutical composition).

The invention further provides a use of a compound of the invention, as described herein above in all aspects and embodiments of a compound of the invention, for the manufacture of a medicament for the treatment of at least one condition, disease or disorder selected from the group consisting of psychiatric disorders, inflammation, oxidation associated conditions, rheumatoid arthritis, cardiovascular diseases, obesity, diabetes and associated disorders and symptoms, emesis and nausea, ischemic/reperfusion injury associated with myocardial, liver or renal diseases, hypoxia/ischemia injury, neuronal damage due to neurological diseases or injury, cancer and resistance to cancer chemotherapy, epilepsy and convulsions, and any condition or symptom associated therefrom.

In another aspect the invention provides a use of a compound of the invention, as described herein above in all aspects and embodiments of a compound of the invention, for the manufacture of a medicament for reduction of oxidative stress.

In yet another aspect the invention provides a use of a compound of the invention, as described herein above in all aspects and embodiments of a compound of the invention, for the manufacture of a medicament for the treatment of any disease, condition or disorder caused by or associated with oxidative stress.

In some embodiments of a use above, said disease, condition or disorder caused or associated with oxidative stress are selected from the group consisting of cancer, oxidative neurological disorders, free radical associated diseases, ischemia, ischemic reperfusion injury, inflammatory diseases, systemic lupus erythematosis, myocardial ischemia or infarction, cerebrovascular accidents, operative ischemia, traumatic hemorrhage, spinal cord trauma, Down's syndrome, Crohn's disease, autoimmune diseases, cataract formation, uveitis, emphysema, gastric ulcers, oxygen toxicity, neoplasia, undesired cellular apoptosis, radiation sickness, and any combinations thereof.

In a further aspect the invention provides a use of a compound according to the invention (as defined in any of the aspects and embodiments of a compound of the invention), for the manufacture of a medicament for the treatment of oxidative associated disease, disorder or condition of the CNS.

In another aspect the invention provides a use of a compound according to the invention (as defined in any of the aspects and embodiments of a compound of the invention), for the manufacture of a medicament for preventing, arresting, or treating neurological damage in a subject suffering from at least one disease, disorder or condition selected from Parkinson's disease, Alzheimer's disease and HIV dementia; autoimmune neurodegeneration, hypoxic or anoxic neuronal damage, respiratory arrest or cardiac arrest, anoxia caused by drowning and brain surgery or trauma and any combinations thereof.

In a further aspect the invention provides a use of a compound according to the invention (as defined in any of the aspects and embodiments of a compound of the invention), for the manufacture of a medicament for the treatment of an ischemic or neurodegenerative disease in the central nervous.

In some embodiments of a use above, said ischemic or neurodegenerative disease is selected from the group consisting of: an ischemic infarct, Alzheimer's disease, Parkinson's disease, and human immunodeficiency virus dementia, Down's syndrome, and heart disease or any combinations thereof.

The invention also provides a method of treating a condition, disease, disorder or symptom associated with inflammation in a subject in need thereof, said method comprising administering to said subject an effective amount of at least one compound of the invention, as described herein above in all aspects and embodiments of a compound of the invention.

The invention further encompasses a method of reduction of oxidative stress in a tissue or an organ of a subject in need thereof, said method comprising administering to said subject an effective amount of at least one compound of the invention, as described herein above in all aspects and embodiments of a compound of the invention.

According to a further aspect the invention provides a method of treating any disease, condition or disorder caused by or associated with oxidative stress a subject in need thereof, said method comprising administering to said subject an effective amount of at least one compound of the invention, as described herein above in all aspects and embodiments of a compound of the invention.

In some embodiments of a method above, said disease, condition or disorder caused by or associated with oxidative stress are selected from the group consisting of cancer, oxidative neurological disorders, free radical associated diseases, ischemia, ischemic reperfusion injury, inflammatory diseases, systemic lupus erythematosis, myocardial ischemia or infarction, cerebrovascular accidents, operative ischemia, traumatic hemorrhage, spinal cord trauma, Down's syndrome, Crohn's disease, autoimmune diseases, cataract formation, uveitis, emphysema, gastric ulcers, oxygen toxicity, neoplasia, undesired cellular apoptosis, radiation sickness, and others.

In a further aspect the invention provides a method for the treatment of oxidative associated disease, disorder or condition of the CNS in a subject, comprising administering to said subject a therapeutically effective amount of a compound of the invention (as defined in any of the aspects and embodiments of a compound of the invention).

In another aspect the invention provides a method for preventing, arresting, or treating neurological damage in a subject suffering from at least one disease, disorder or condition selected from Parkinson's disease, Alzheimer's disease and HIV dementia; autoimmune neurodegeneration, hypoxic or anoxic neuronal damage, respiratory arrest or cardiac arrest, anoxia caused by drowning and brain surgery or trauma, comprising administering to said subject a therapeutically effective amount of a compound of the invention (as defined in any of the aspects and embodiments of a compound of the invention).

In a further aspect the invention provides a method of treating an ischemic or neurodegenerative disease in the central nervous system of a subject, comprising administering to the subject a therapeutically effective amount of a compound of the invention (as defined in any of the aspects and embodiments of a compound of the invention).

In some embodiments of a method above, said ischemic or neurodegenerative disease is selected from the group consisting of: an ischemic infarct, Alzheimer's disease, Parkinson's disease, and human immunodeficiency virus dementia, Down's syndrome, and heart disease or any combinations thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows the effects of HU-475 (a compound of the invention 1, 3 and 10 nmol, n=8-9 animals/group) and vehicle (n=7) microinjected into the dorsolateral periaqueductal gray of rats submitted to the elevated plus maze. Data expressed as means±SEM of the percentage of entries onto the open arms. * indicates significant difference from vehicle (p<0.05).

FIG. 2 shows the effects of HU-475 (a compound of the invention 1, 3 and 10 nmol, n=8-9 animals/group) and entries onto vehicle (n=7) microinjected into the dorsolateral periaqueductal gray of rats submitted to the elevated plus maze. Data expressed as means±SEM of the percentage of time spent in the open arms. + indicates a trend (p<0.1) for a difference from vehicle.

FIG. 3 shows the effects of HU-474 (a compound of the invention 1, 3 and 10 mg/kg n=6-8 animals/group) and vehicle (n=7) in mice tested in the elevated plus maze. Data expressed as means±SEM of the percentage of time in the open arms. * indicates significant difference from vehicle.

FIG. 4 shows the effects of HU-474 (a compound of the invention 1, 3 and 10 mg/kg n=6-8 animals/group) and vehicle (n=7) in mice tested in the elevated plus maze. Data expressed as means±SEM of the percentage of entries onto the open arms.

FIG. 5 shows the effects of HU-474 (a compound of the invention 1, 3 and 10 mg/kg n=6-8 animals/group) and vehicle (n=7) in mice immobility time (s) showed by mice tested in the FST. Data expressed as means±SEM. * indicates significant difference from vehicle.

FIG. 6 shows the effects of HU-474 (3 and 10 mg/kg i.p.) in mice on the impairment of PPI induced by MK-801 (M0.5 mg/kg). Results are expressed as means±SEM. * indicates significant difference from vehicle-vehicle, # significant difference from vehicle-MK group.

FIG. 7 shows the effect of HU-485 (1, 3 and 10 mg/kg, n=5, 5 and 6 animals, respectively) in Swiss male mice tested in the forced swim test (FST, upper panel) and elevated plus maze (EPM). Data represents the means±SEM of immobility time in the FST and the % of open arm entries and time spent in these arms of the EPM. * indicates difference from vehicle.

FIG. 8 shows the effect of CBD (15, 30 and 60 mg/kg, n=10 animals/group) in Swiss male mice tested in the pre-pulse inhibition model. Data represents the means±SEM. * indicates difference from vehicle-vehicle. + indicates difference from vehicle-amphetamine group (p<0.05).

FIG. 9 shows the effect of HU-485 (3, 10 and 30 mg/kg, n=7-8 animals/group) in Swiss male mice tested in the pre-pulse inhibition model. Data represents the means±SEM. * indicates difference from vehicle-vehicle group (p<0.05).

### DETAILED DESCRIPTION OF EMBODIMENTS

### Example 1: Synthesis of Compound 8 (HU-485)

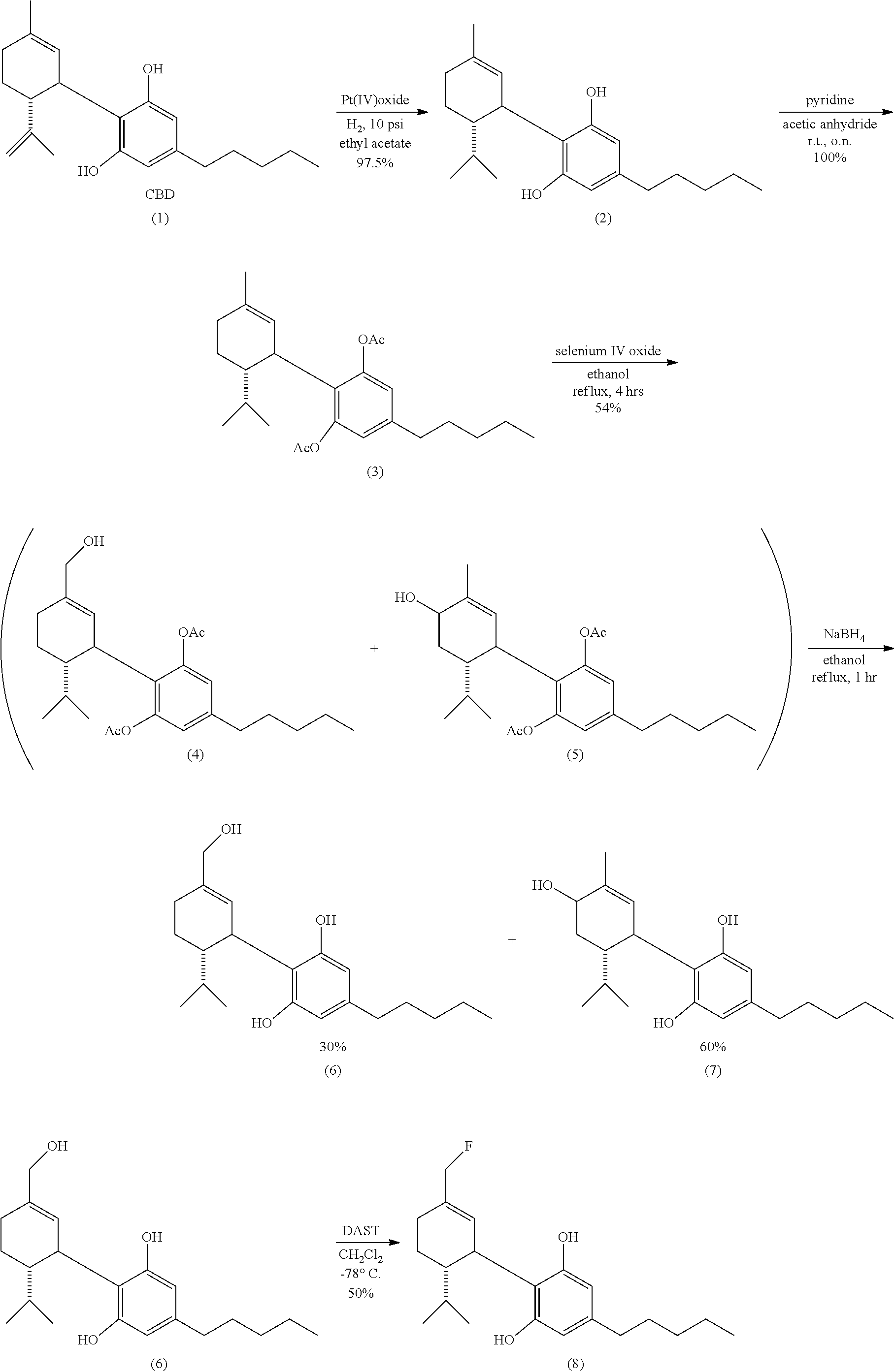

### Step A

Cannabidiol (CBD, 1) was isolated from hashish following the procedure described by Gaoni and Mechoulam (1971). 1H-NMR (CDCl3, 300 MHz): δ 6.10-6.30 (br, 2H, ArH), 5.95-6.05 (br, 1H, OH), 5.57 (s, 1H), 4.91-5.01 (br, 1H, OH), 4.66 (s, 1H), 4.55 (s, 1H), 3.91 (br s, 1H), 2.39-2.46 (m, 3H), 2.01-2.21 (br t, 2H), 1.79 (s, 3H), 1.66 (s, 3H), 1.53-1.61 (m, 2H), 1.22-1.33 (m, 7H), 0.90 (t, J=7.5 Hz, 3H). GC-MS m/z: 314, 300, 285, 270, 260. Melting point, 62° C. [α]20D=−56° in CHCl3.

CBD (1, 0.544 g, 1.73 mmol) was hydrogenated in ethyl acetate (10 ml), over a PtO2 catalyst (0.021 g), at 10 psi, for 2 min. The mixture was purified by silica gel chromatography, using 1% ether-petroleum ether as an eluent to obtain Compound 2 (0.534 mg., 97.5%). 1H-NMR (CDCl3, 300 MHz): δ 6.10-6.30 (br, 2H, ArH), 5.95-6.05 (br, 1H, OH), 5.57 (s, 1H), 4.90-5.10 (br, 1H, OH), 3.87 (br s, 1H), 2.40-2.46 (m, 3H), 2.00-2.21 (br t, 2H), 1.79 (s, 3H), 1.66 (s, 3H), 1.53-1.63 (m, 2H), 1.22-1.33 (m, 7H), 0.90 (t, J=7.5 Hz, 6H). GC-MS m/z: 316, 302, 287, 272, 262. [α]20D=−58° in CHCl3. Compound 2 was previously reported by Ben-Shabat et al., 2006.

### Step B

Compound 2 (0.534 g, 1.689 mmol) was dissolved in pyridine (3 ml) and acetic anhydride (3 ml) and the reaction was stirred overnight at room temperature. Then the solution was poured onto iced water (50 ml) and extracted with ether. The combined organic extracts were washed successively with 1 M HCl, aqueous sodium bicarbonate and brine, dried over MgSO4, filtered and evaporated to dryness to obtain Compound 3 (0.677 g, 100%). GC-MS m/z: 400, 386, 371, 356, 346.

### Step C

Compound 3 (0.677 g, 1.69 mmol) was dissolved in ethanol (4 ml) and selenium oxide (SeO2, 0.564 g, 5.08 mmol) was added. The mixture was refluxed for 4 h and monitored by TLC. The ethanol was removed under pressure and the residue was diluted with water and extracted several times with ether, dried over MgSO4, filtered and evaporated to dryness. The residue was chromatographed on silica gel with (20% ether-petroleum ether) to give mixtures of Compounds 4 and 5 (0.471 g, 67%).

### Step D

Mixtures of Compounds 4 and 5 (0.471 g, 1.323 mmol) was dissolved in ethanol (125 ml), NaBH4 (0.056, 1.505 mmol) was added and the reaction was refluxed for 1 h. The ethanol was removed under reduced pressure, the residue was diluted with water (180 ml) and the solution was extracted with ether. The combined organic extracts were washed with brine, dried on MgSO4 and filtered. Removal of the solvents under reduced pressure afforded a residue that was chromatographed on silica gel (30% ether-petroleum ether) to provide Compound 6 (0.131 g, 30%) and Compound 7 (0.263 g, 60%). Compound 6: 1HNMR (CDCl3, 300 MHz): δ 6.225 (s, 2H, Ar), 5.687 (s, 1H, olefin), 3.899 (m, 1H, benzyl), 2.422 (t, 1H, allyl), 2.222 (t, 2H, benzyl), 1.858 (m, 2H), 1.623 (s, 6H, allyl CH3), 1.545 (m, 2H), 1.289 (m, 6H), 0.889 (t, 9H, terminal CH3). GC-MS m/z: 548 (silylation), 506, 478, 466, 416, 390. [α]20D=−67.5° in CHCl3. Analysis calculated for C21H32O3: 332.21949, found 332.2321. Compound 7: 1HNMR (CDCl3, 300 MHz): δ 6.22 (s, 2H, Ar), 5.69 (s, 1H, olefin), 3.90 (m, 1H, benzyl), 3.69 (t, 1H), 2.42 (t, 1H, allyl), 1.828 (s, 3H), 1.62 (s, 6H, allyl CH3), 1.54 (m, 2H), 1.29 (m, 6H), 0.89 (t, 9H, terminal CH3). GC-MS m/z: 548 (silylation), 506, 476, 458, 415, 392. [α]20D=−69.1° in CHCl3. Analysis calculated for C21H32O3: 332.2392, found 332.2401.

### Step E

To a stirred solution of Compound 6 (500 mg, 1.506 mmol) in CH2Cl2 (12 mL) at −78° C. was added DAST (0.238 mL, 1.807 mmol, 1.2 equiv) at −78° C., stirred for 1 hr at the same temperature under N2 atmosphere, and the resulting mixture was slowly warmed to room temperature and stirred for 4 hrs monitoring by TLC. The reaction was carefully quenched with saturated Na2CO3 solution and extracted with CH2Cl2 (6 ml×2). The combined extracts were washed with 5 ml H₂O and dried over MgSO4. After evaporation of the solvent under reduced pressure, the resulting residue was chromatographed using silica gel (1% ether-petroleum ether) to give Compound 8 as light yellow oil (250 mg, 50%). 1HNMR (CDCl3, 300 MHz): δ 6.200 (s, 2H, Ar), 5.822 (s, 1H, olefin), 4.752 (d, 2H, CH2F), 3.962-3.923 (m, 1H, benzyl), 2.567-2.484 (td, 1H, J=13.3, 2.7 Hz, allyl), 2.435-2.384 (t, 2H, J=7.5 Hz, benzyl), 1.882-1.734 (m, 2H), 1.660 (s, 6H, allyl CH3), 1.584-1.487 (m, 2H), 1.285-1.248 (m, 6H), 0.886-0.843 (t, 6H, J=6.3 Hz, terminal CH3); GC-MS m/z: 478 (silylation), 406, 334, 315, 299, 283. [α]20D=−55.3° in CHCl3, Analysis calculated for C21H31FO2: 334.2312, found 334.2431.

### Example 2: Synthesis of Compound 9 (HU-487)

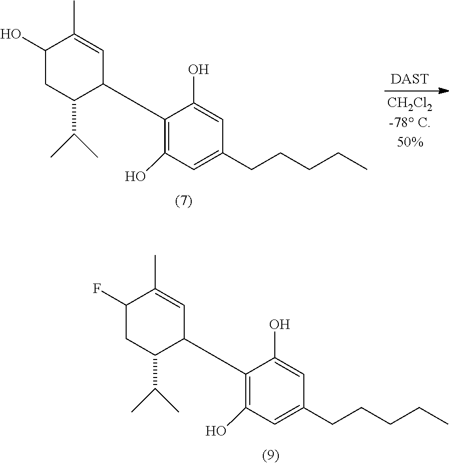 [SMILES: CCCCCc1cc(O)c(C2C=C(C)C(O)C[C@H]2C(C)C)c(O)c1 ; "DAST" ; CCCCCc1cc(O)c(C2C=C(C)C(F)C[C@H]2C(C)C)c(O)c1]

Compound 9 was synthesized starting from Compound 7 (as prepared in Example 1) by the same procedure as Example 1, Step E (80%). 1HNMR (CDCl3, 300 MHz): δ 6.225 (s, 2H, Ar), 5.687 (s, 1H, olefin), 3.899 (m, 1H, benzyl), 3.79 (dt, 1H), 2.422 (t, 1H, allyl), 1.828 (s, 3H), 1.623 (s, 6H, allyl CH3), 1.545 (m, 2H), 1.289 (m, 6H), 0.889 (t, 9H, terminal CH3). GC-MS m/z: 478 (silylation), 459, 387, 315, 299, 283. [α]20D=−59.8° in CHCl3, Analysis calculated for C21H31FO2: 334.2312, found 334.2321.

### Example 3: Synthesis of Compound 19 (HU-559a)

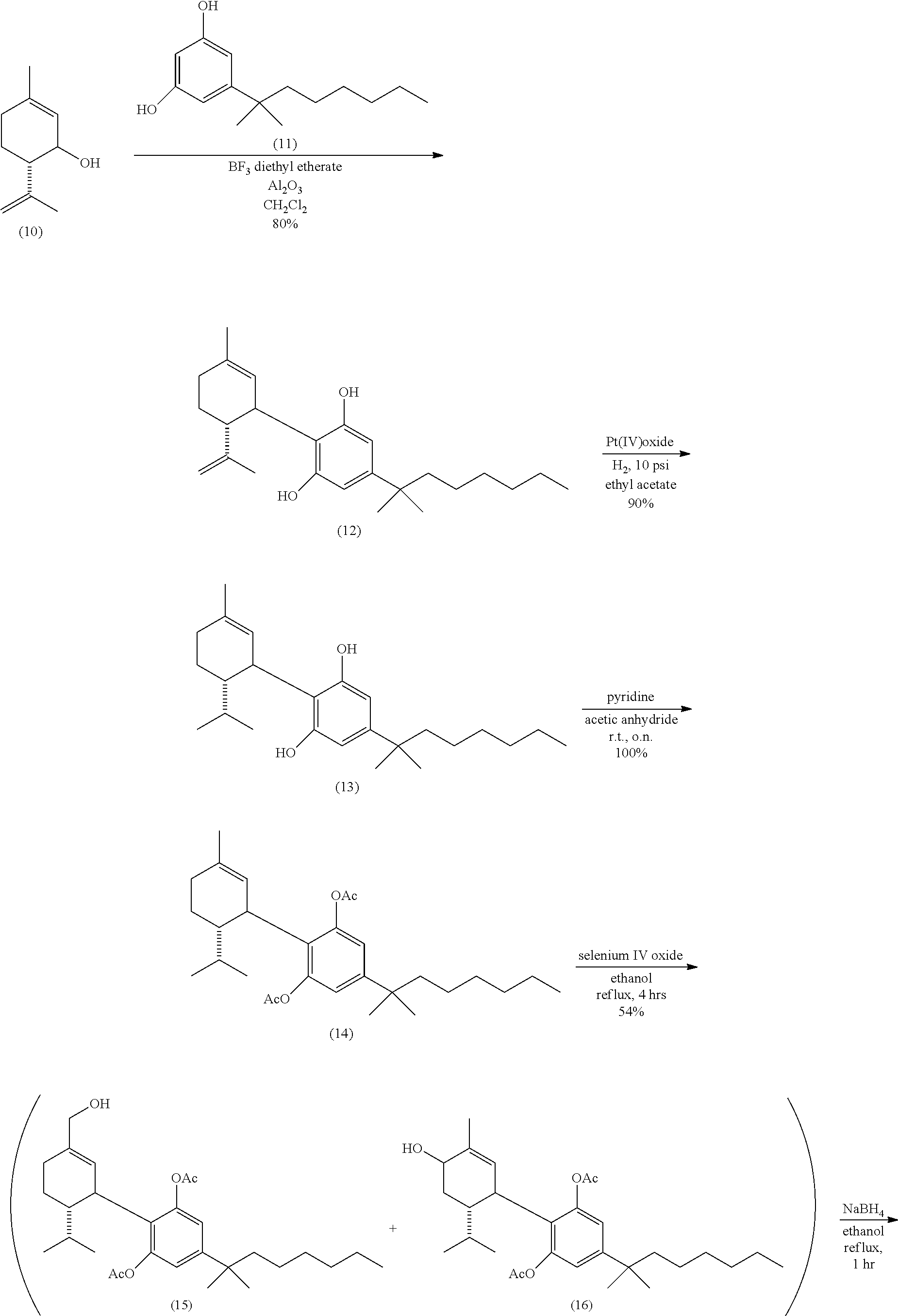

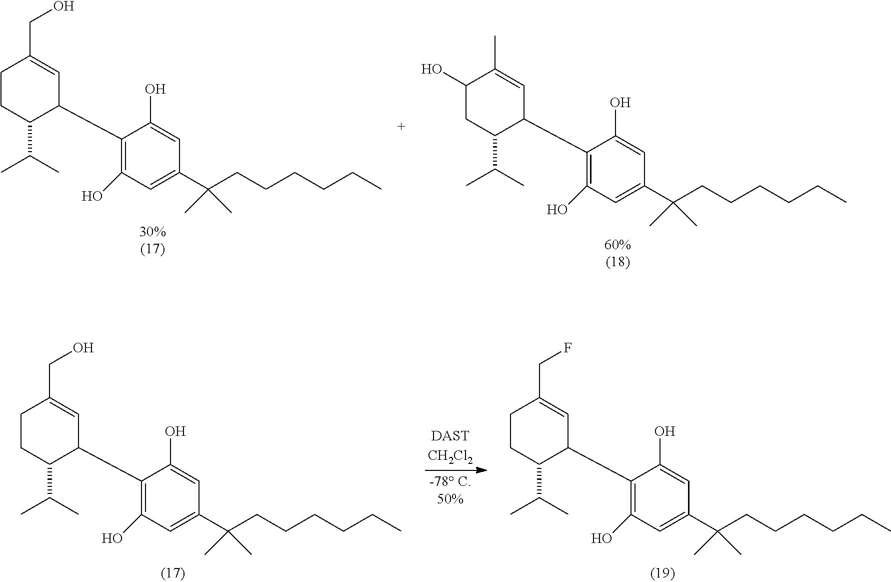 [SMILES: CCCCCCC(C)(C)c1cc(O)c(C2C=C(CO)CC[C@H]2C(C)C)c(O)c1 ; CCCCCCC(C)(C)c1cc(O)c(C2C=C(C)C(O)C[C@H]2C(C)C)c(O)c1 ; CCCCCCC(C)(C)c1cc(O)c(C2C=C(CO)CC[C@H]2C(C)C)c(O)c1 ; CCCCCCC(C)(C)c1cc(O)c(C2C=C(CF)CC[C@H]2C(C)C)c(O)c1]

### Step A

Basic aluminium oxide (15.6 g) was added to dry dichloromethane (150 ml). To this suspension BF3.OEt2 (2.3 ml) was added under nitrogen. The mixture was stirred for 15 min at room temperature under N2 atmosphere. To the solution was added (+)-p-mentha-1,8-diene-3-ol ((+)-isopiperitenol) 10 (950 mg, 6.25 mmol) and dimethylhepty resorcinol 11 (1.45 g, 6.25 mmol) in dry dichloromethane (50 ml) and the reaction mixture was quenched within sec with 10% aqueous solution of sodium bicarbonate (50 ml). The organic layer was separated and the aqueous layer was further extracted with dichloromethane. The combined dichloromethane solution was extracted with water, brine, dried on Na2SO4 and evaporated to give an red oil. This oil was purified by silica gel column chromatography, using petroleum ether and ether as an eluent to obtain Compound 12 (1.86 g, 80%). 1H-NMR (CDCl3, 300 MHz): δ 6.250-6.358 (br, 2H, ArH), 5.90-6.050 (br, 1H, OH), 5.560 (s, 1H), 4.656 (s, 1H), 4.545 (s, 1H), 4.556 (s, 1H), 3.850 (br, 1H), 2.050-2.300 (m, 2H), 1.794 (s, 3H), 1.635 (s, 3H), 1.454-1.505 (m, 2H), 1.234-1.335 (m, 3H), 1.208 (br s, 12H), 0.950-1.050 (br, 2H), 0.832 (t, J=7.5 Hz, 3H); GC-MS m/z: 370, 302, 287, 249, 217, 202, 187 [α]20D=−65.1° in CHCl3

### Step B

Compound 13 was prepared by the same procedure as reported in Example 1, Step A above (90%). 1H-NMR (CDCl3, 300 MHz): δ 6.250-6.358 (br, 2H, ArH), 5.90-6.050 (br, 1H, OH), 05.560 (s, 1H), 4.556 (s, 1H), 3.850 (br, 1H), 2.050-2.300 (m, 2H), 1.794 (s, 3H), 1.635 (s, 3H), 1.454-1.505 (m, 2H), 1.234-1.335 (m, 3H), 1.208 (br s, 12H), 0.951-1.062 (br s, 3H), 0.891 (t, J=7.5 Hz, 6H); GC-MS m/z: 372, 304, 289, 251, 219, 204, 187 [α]20D=−66.2° in CHCl3.

### Step C

Compound 14 was prepared by the same procedure as reported in Example 1, Step B above (100%). 1H-NMR (CDCl3, 300 MHz): δ 7.250-7.358 (br, 2H, ArH), 5.90-6.050 (br, 1H, OH), 5.560 (s, 1H), 4.556 (s, 1H), 3.850 (br, 1H), 2.280 (s, 6H), 2.050-2.300 (m, 2H), 1.635 (s, 3H), 1.454-1.505 (m, 2H), 1.236-1.337 (m, 3H), 1.208 (br s, 12H), 0.950-1.050 (br s, 1H), 0.832 (t, J=7.5 Hz, 9H); GC-MS m/z: 456, 304, 289, 251, 219, 204, 187 [α]20D=−70.2° in CHCl3

### Step D

Compounds 15 and 16 were prepared by the same procedure as reported in Example 1, Step C above.

### Step E

Compounds 17 and 18 were prepared by the same procedure as reported in Example 1, Step D above (Compound 17, 30%; Compound 18, 60%). Compound 17: 1H-NMR (CDCl3, 300 MHz): δ 6.335 (s, 2H, Ar), 5.863 (s, 1H), 4.652 (s, 1H), 3.920-3.889 (d, 1H, J=9.3 Hz, benzyl), 2.498-2.433 (m, 1H, allyl), 2.228 (br s, 2H), 2.064-1.715 (m, 2H), 1.648-1.428 (m, 9H), 1.312-1.168 (m, 12H), 0.863-0.817 (t, 9H, J=6.5 Hz, terminal CH3); [α]20D −62.70 in CHCl3; GC-MS m/z: 388, 371, 370, 357, 302, 289, 285, 251, 235, 189; Analysis calculated for C25H40O3: 388.28210, found 388.2825.

Compound 18: 1HNMR (CDCl3, 300 MHz): δ 6.225 (s, 2H, Ar), 5.687 (s, 1H, olefin), 3.899 (m, 1H, benzyl), 3.69 (t, 1H), 2.422 (t, 1H, allyl), 1.828 (s, 3H), 1.623 (s, 6H, allyl CH3), 1.545 (m, 2H), 1.433-1.289 (m, 14H), 0.889 (t, 9H, terminal CH3). GC-MS m/z: 388, 372, 370, 357, 302, 288, 284, 251, 234, 189. [α]20D −64.7° in CHCl3, Analysis calculated for C25H40O3: 388.28210, found 388.2715.

### Step F

Compound 19 was prepared by the same procedure as reported in Example 1, Step E above (50%). 19 1HNMR (CDCl3, 300 MHz): δ 6.300 (s, 2H, Ar), 5.742 (s, 1H, olefin), 4.862 (d, 2H, CH2F), 3.972-3.973 (m, 1H, benzyl), 2.577-2.487 (td, 1H, J=13.3, 2.7 Hz, allyl), 2.444-2.394 (t, 2H, J=7.5 Hz, benzyl), 1.902-1.756 (m, 2H), 1.678 (s, 6H, allyl CH3), 1.594-1.497 (m, 2H), 1.295-1.258 (m, 6H), 0.906-0.913 (t, 3H, J=6.3 Hz, terminal CH3). GC-MS m/z: 390, 371, 369, 356, 339, 333. [α]20D −62.70 in CHCl3, Analysis calculated for C25H39FO2: 390.28210, found 390.2825.

### Example 4: Synthesis of Compound 20

Compound 20 was synthesized starting from Compound 18 (as prepared in Example 3) by the same procedure as Example 3, Step F (50%). GC-MS m/z: 390, 372, 370, 357, 339. [α]20D −64.7° in CHCl3, Analysis calculated for C25H39FO2: 390.28210, found 390.2715. Furthermore, Compound 20's two aromatic free hydroxyl groups can be further protected by an acetyl group under commonly used hydroxyl protection chemistry, for example, see Example 1, step B.

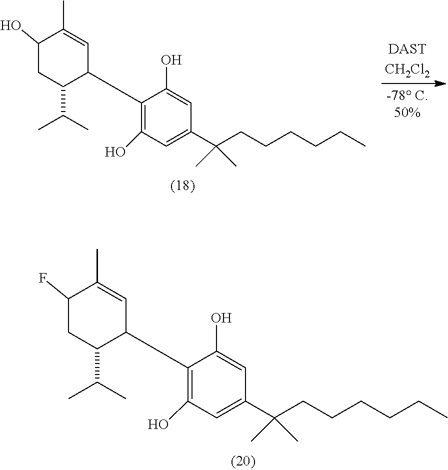 [SMILES: CCCCCCC(C)(C)c1cc(O)c(C2C=C(C)C(O)C[C@H]2C(C)C)c(O)c1 ; CCCCCCC(C)(C)c1cc(O)c(C2C=C(C)C(F)C[C@H]2C(C)C)c(O)c1]

### Example 5: Fluorination of cannabidiol (HU-474)

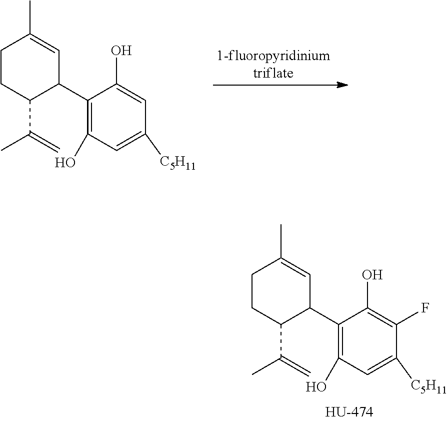

To a solution of cannabidiol (942 mg, 3 mmol) in dry CH2Cl2 (42 mL) was added 1-fluoropyridinium triflate (742 mg, 3 mmol) and the reaction mixture was stirred at ambient temperature overnight. After dilution with CH2Cl2 the mixture was washed with saturated aqueous solution of NaHCO₃. The organic layer was separated, dried over MgSO4 and evaporated. The oil obtained was chromatographed on a silica gel column (75 g). Elution with 2% ether in petroleum ether gave the compound 4′-fluoro-cannabidiol (HU-474) as a solid (300 mg, 27%) m.p. 59-61° C. 1H NM R (300 MHz, CDCl3) δ 6.17 (1H, s, arom.) 5.52 (1H, s), 4.56 (1H, s), 4.44 (1H, s), 3.92 (1H, s), 2.50 (2H, b), 2.19-2.05 (2H, b), 1.77 (3H, s), 0.86 (3H, t). MS, m/e=332 (M+).

### Example 6: Fluorination of Cannabidiol Diacetate (HU-475)

### Step A

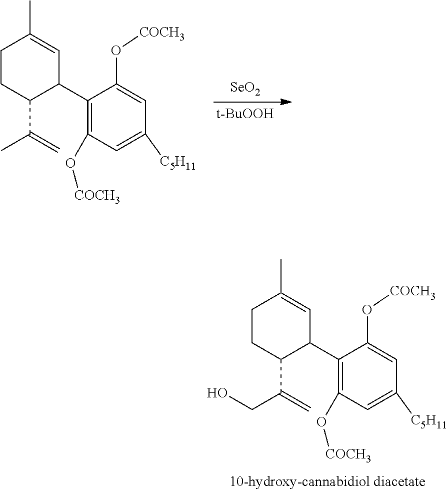

To a suspension of SeO2 (219 mg, 2 mmol) and t-BuOOH (2.8 mL, 70% in water) in CH2Cl2 (7 mL) was added a solution of cannabidiol diacetate (2 g, mmol) in CH2Cl2 (10 mL). After stirring the mixture at ambient temperature overnight, it was washed with saturated aqueous solution of NaHCO₃ followed by a saturated solution of NaHSO3. The organic layer was separated, dried over MgSO4, filtered and evaporated. The oil obtained was purified on a silica gel column (50 g). Elution with 13% ether in petroleum ether gave the required compound 10-hydroxy-cannabidiol diacetate (670 mg, 40%) as oil. 1H NMR (300 MHz, CDCl3), b 6.7 (2H, s), 5.17 (1H, s), 4.99, (1H, s), 4.87 (1H, s), 3.70-3.80 (2H m), 3.53-3.58 (1H, m), 2.52-2.57 (2H, t J=7.6 Hz), 2.39-2.48 (1H d J=5.1 Hz), 2.18 (6H, s), 2.06 (1H, s), 2.0 (1H, s), 1.58 (1H, s), 1.25-1.31 (4H, m), 0.86-0.90 (3H t, J=6.45 Hz).

### Step B

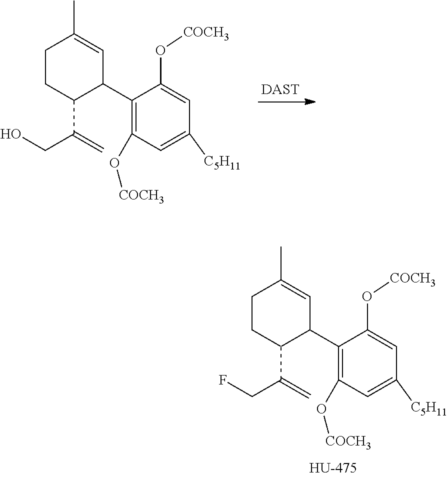

The alcohol (414 mg, 1 mmol) in dry CH2Cl2 (4 mL) was added under N2 atmosphere to an ice-cold solution of DAST (0.18 mL, 1.5 mmol). After 15 min. at 0° C. solid Na2CO3 (125 mg, 1 mmol) was added. The organic phase was then washed twice with cold 1 M aqueous Na2CO3 solution, followed by water. The organic layer was separated, dried over MgSO4, filtered and evaporated. The resulting crude material was purified on a silica gel column (20 g) using 10% ether in petroleum ether to provide the fluorinated product 10-fluoro-cannabidiol diacetate (HU-475) (77.5 mg, 18.6%). 1H NMR (300 MHz, CDCl3) δ 6.74 (2H, s), 5.21 (1H, s), 5.01 (1H, s), 4.87 (1H, s), 4.60 (1H, s), 4.50 (1H, s), 3.6 (1H, b), 2.73 (1H, t), 2.57 (2H, t), 2.21 (6H, s), 2.08-1.59 (8H, ms), 1.32 (3H, s), 0.90 (3H, t). MS, m/e=416 (M+).

### Example 7: In Vivo Effect of HU-474 in Mice and HU-475 in Rats

### 1. Animals

Male Wistar rats (220-250 g) and Swiss mice (25-30 g) originated from the Central Animal Farm of the School of Medicine of Ribeirio Preto (FMRP-USP) were maintained in groups of five animals per box (41×33×17 cm) in a temperature controlled room (24±2° C.) with a 12×12 h light-dark cycle. They received water and food ad libitum throughout the study period.

### 2. Compositions

HU-474 (1, 3 and 10 mg/kg) was administered intraperitoneally (IP) in mice at 10 mL/kg volume and HU-475 (1, 3 and 10 nmol) was injected intra-dIPAG in rats. Both drugs were dissolved in 2% Tween 80 in sterile saline.

### 3. Stereotaxic Surgery (HU-475)

Rats were submitted to a stereotaxic surgery to unilaterally implant cannulae (9.0 mm, 0.6 mm OD) into the dIPAG (coordinates: lateral: −1.9 mm; depth: −4.3 mm; angle: 16° from lambda; Paxinos and Watson, 2005), fixed to the skull with acrylic cement (Campos & Guimarães, 2008). The surgeries were performed under anesthesia with tribromoethanol 2.5% (10.0 mL/kg, IP) and immediately after the animals received Veterinary Pentabiotic (0.2 mL, intramuscular) and analgesic (Banamine, 1.0 mL/kg, subcutaneous) to prevent infections and decrease post-surgical pain. After surgery, animals underwent a recovery period of 5-7 days before the behavioral tests.

### 4. Microinjection (HU-475)

Animals received unilateral microinjections of vehicle or HU-475 into the dIPAG before being submitted to the behavioral tests. To this aim, microneedles (10.0 mm, 0.3 mm OD), connected to a microsyringe (Hamilton, USA, 10 mL) through a segment of polyethylene (P10) were inserted into the guide cannulae. Solutions were injected with the help of an infusion pump (KD Scientific, USA). A 0.2 μL solution volume was injected over 1 min. After the injections, the needles remained inserted in the cannulae for additional 30 seconds to prevent drug reflux (Campos & Guimarães, 2008).

### 5. Apparatus

### 5.1 Elevated Plus-Maze (EPM) Rats

The wood EPM used to perform the experiments was located in a sound attenuated and temperature controlled room (23° C.), with one incandescent light (40 W) placed 1.3 m away from the maze. The apparatus consists of two opposing open arms (50×10 cm) without side walls, perpendicular to two enclosed arms (50×10×40 cm), with a central platform common to all arms (10×10 cm). The apparatus is elevated 50 cm above the ground and has an acrylic edge (1 cm) in the open arms to prevent animal falls. In this model, rodents naturally avoid the open arms, exploring more extensively the enclosed arms. Anxiolytic drugs increase the exploration in open arms without affecting the number of enclosed arms entries, which is usually used to assess general exploratory activity (File, 1992). Ten minutes after the last injection the animal was placed on the central platform of the maze with the head facing one of the enclosed arms. The test lasted for 5 min and was recorded. The animal behavior was analyzed with the help of the Anymaze Software (version 4.5, Stoelting). This software indicates the location of the animal in the EPM and automatically calculates the percentage of entries (Peo) and time spent in the open arms (Pto) and the number of entries in the enclosed arms (EA). Animals were only considered to enter an open or enclosed arm when 90% of their bodies were inside the region. All experiments were performed in the morning period (8 to 12 a.m.).

### 5.2 Elevated Plus-Maze (EPM) Mice

Similar to 5.1 except that the each arm measured 30×5 cm. 5.3 Forced Swimming Test (FST) Mice

Animals were individually submitted for 6 min of forced swimming in glass cylinders (height 25 cm, diameter 17 cm) containing 10 cm of water. The mice were videotaped and the immobility time (characterized by slow movements necessary to avoid drowning) was measured during the last 4-min period. The water was changed after each trial to maintain the temperature at 23-25° C. and to prevent the influence of alarm substances (Zanelati et al., 2010).

### 6. Histology—Rats

After the behavioral tests animals were anesthetized with chloral hydrate 4% (10 mL/kg) and perfused with saline 0.9%. Brains were removed and kept in formalin solution 10% for 3-7 days. Soon after, brains were cut into 50-μm thick sections in a cryostat (Cryocut 1800). The injection sites were identified in diagrams from the Paxinos and Watson's atlas (Paxinos and Watson, 2005). Rats receiving injections outside the aimed area were included in a separate group (out group).

### 7. Statistical Analysis

Results from HU-475 and HU-474 tests in the elevated plus maze were analyzed by Kruskal-Wallis followed by Mann-Whitney tests. Data from animals tested in the FST were analyzed by one-way ANOVA followed by Duncan test.

### Results.

### HU-475.

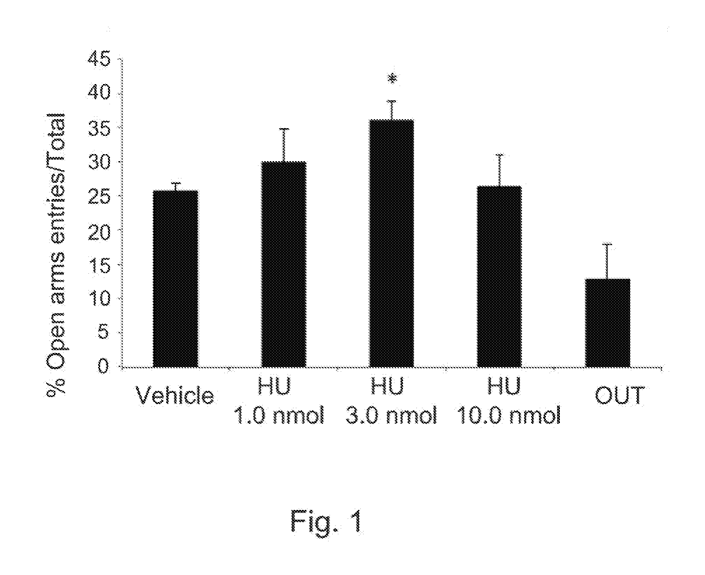

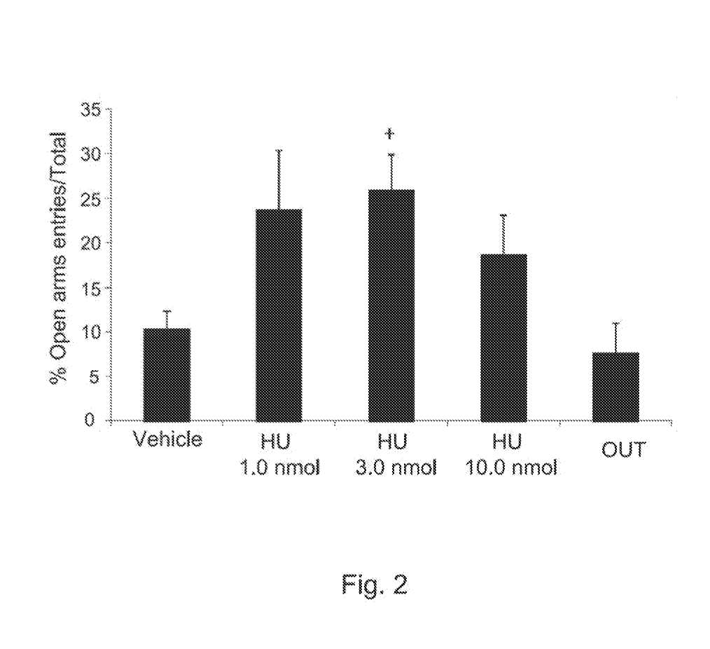

The drug increased the percentage of entries (X2=9.66, DF=4, p<0.05, FIG. 1) and tended to do the same (X2=8.5, DF=4, p=0.075, FIG. 2) with the percentage of time spent into the open arms. No effect was found in the number of enclosed arm entries.

FIG. 1 shows the effects of HU-475 (1, 3 and 10 nmol, n=8-9 animals/group) and vehicle (n=7) microinjected into the dorsolateral periaqueductal gray of rats submitted to the elevated plus maze. Results from animals that received the dose of 3 nmol outside the target region are shown in the OUT group (n=6). Data expressed as means±SEM of the percentage of entries onto the open arms. * indicates significant difference from vehicle (p<0.05). FIG. 2 shows the effects of HU-475 (1, 3 and 10 nmol, n=8-9 animals/group) and vehicle (n=7) microinjected into the dorsolateral periaqueductal gray of rats submitted to the elevated plus maze. Results from animals that received the dose of 3 nmol outside the target region are shown in the OUT group. Data expressed as means±SEM of the percentage of time spent in the open arms. + indicates a trend (p<0.1) for a difference from vehicle.

### HU-474.

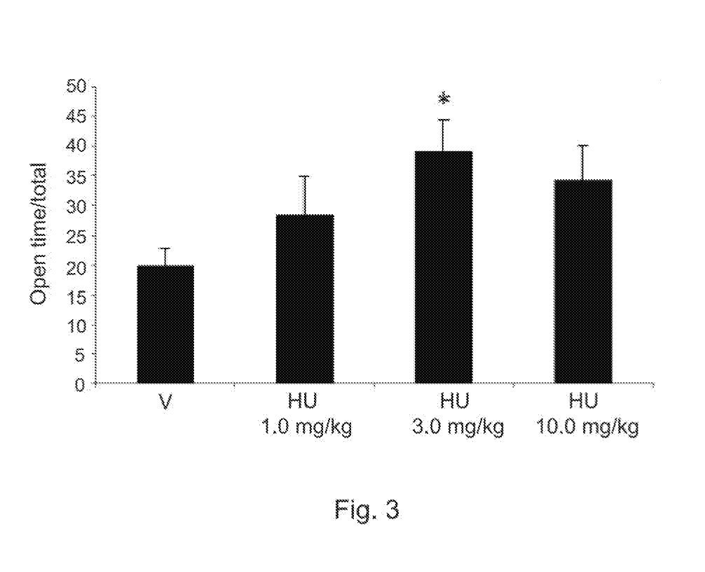

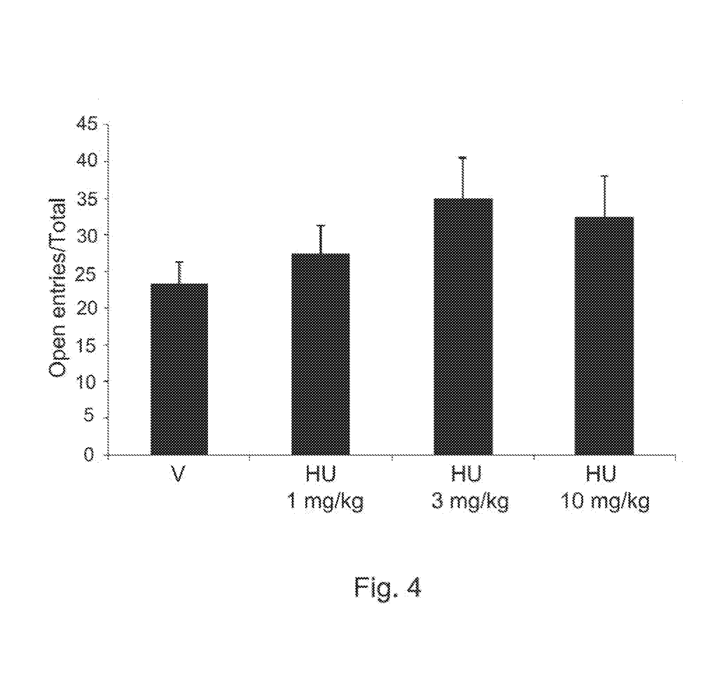

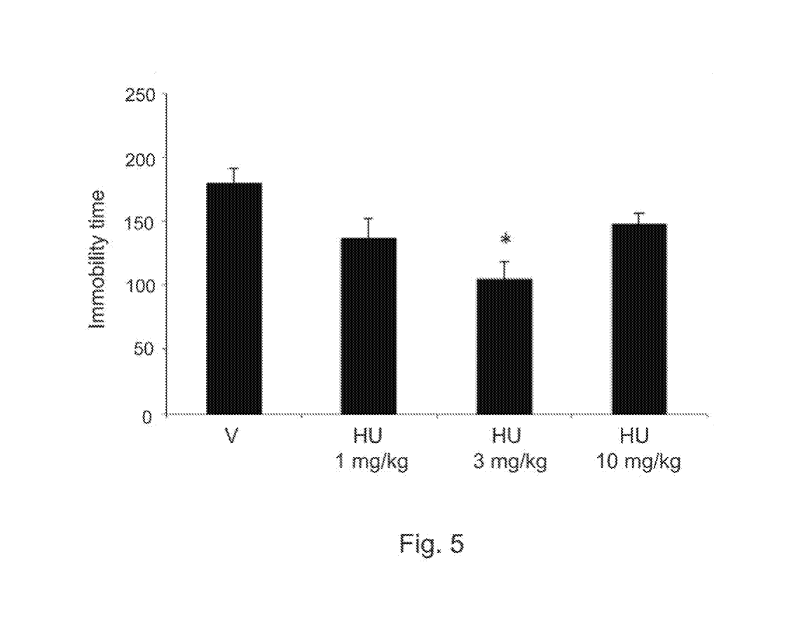

The drug increased the percentage of time spent in the open arms of the pEPM (X2=8.13, DF=3, p<0.05, FIG. 3. No effect was found in the percentage of entries onto these same arms (FIG. 4) and the number of enclosed arms entries. The drug also decreased immobility time in the FST (F3,2=4.06, p=0.019, FIG. 5) at the dose of 3 m/kg. The doses of 1 and 10 mg/kg were ineffective. FIG. 3 shows the effects of HU-474 (1, 3 and 10 mg/kg n=6-8 animals/group) and vehicle (n=7) in mice tested in the elevated plus maze. Data expressed as means±SEM of the percentage of time in the open arms. * indicates significant difference from vehicle. FIG. 4 shows the effects of HU-474 (1, 3 and 10 mg/kg n=6-8 animals/group) and vehicle (n=7) in mice tested in the elevated plus maze. Data expressed as means±SEM of the percentage of entries onto the open arms. FIG. 5 shows the effects of HU-474 (1, 3 and 10 mg/kg n=6-8 animals/group) and vehicle (n=7) in mice immobility time (s) showed by mice tested in the FST. Data expressed as means±SEM. * indicates significant difference from vehicle.

Discussion (Comparative Results with CBD)

Intra-dIPAG injection of HU-475 increased exploration of the open arms of the EPM without changing the number of enclosed arm entries. This indicates an anxiolytic-like effect (File, 1991) and was similar to that produced by CBD using the same paradigm, including a bell-shaped dose-response curve. However, the effective dose of CBD was 30 nmol (doses tested: 15, 30 and 60 nmol), same dose produced an anxiolytic-like effect in the Vogel punished licking test (Campos & Guimarães, 2008). In this model, therefore, HU-475 was 10 times more potent that CBD.

Systemic administration of HU-474 induced anxiolytic-like effects in mice tested the EPM with a characteristic bell-shaped dose-response curve. The effective dose was 3 mg/kg. In comparison with CBD, Onaivi et al. (1990) in a study conducted with a different mice strain (ICR), observed similar anxiolytic effects at the doses of 1 and 10 mg/kg i.p. (with the 10 mg/kg of CBD being more effective). HU-474 also decrease immobility time in mice tested in the forced swimming test, a model sensitive to antidepressant drugs. CBD also produced an antidepressant-like effect in Swiss mice tested in this model at the dose of 30 mg/kg i.p. (doses tested 3, 10, 30 and 100 mg/kg). Therefore, in this model HU-474 was 10 times more potent than CBD.

### Example 8: Pre-Pulse Inhibition Test (HU-474)

### 1. Animals

The experiments were performed using male C57BL/6J mice weighting 25-30 g. The animals were maintained throughout the experimental period under standard laboratory conditions with free access to water and food will be used.

### 2. Compositions

HU-474 (3 and 10 mg/kg) was dissolved in 2% Tween 80 in sterile saline (vehicle). MK-801 (a NMDA antagonist, 0.5 mg/kg, Sigma, USA) was dissolved in saline. Drugs were administered intraperitoneally (ip) at 10 mL/kg volume.

### 3. Experimental Procedure

The animals (n=9-11/group) received i.p. administration of vehicle or HU-474 (3 and 10 mg/kg) followed, 30 minutes later, by saline or MK-801 (0.5 mg/kg), resulting in the following experimental groups: vehicle+saline, HU 10+saline, vehicle+MK-801, HU 3+MK-801, HU 10+MK-801. The animals were submitted to PPI test 20 minutes after the last drug injection.

### 4. Pre-Pulse Inhibition (PPI)

The PPI was carried out in three consecutive steps. The first consisted of an acclimation period during which no stimulus was presented. In the second step, called habituation, only the stimulus that triggers the startle (pulse) was presented. The step that assessed the inhibition of startle response pulse consisted of 64 random presentations of the different stimuli: (i) pulse (white noise) 105 dB at 20 ms, (II) pre-pulse (pure tone frequency of 7 kHz) 80, 85 and 90 dB at 10 ms, (III) followed by pre-pulse 100 ms interval between them and (IV) zero (no stimulus). During this session the stimuli are presented at regular intervals of 30 s, 8 presentations of each stimulus. The percentage of the PPI was expressed as the percent inhibition of startle amplitude in response to multiple presentations of the pulse preceded by pre-pulse (PP), depending on the amplitude of the response only to the pulse (P), which was obtained in the following formula: % PPI=100−((PP/P)×100). Using this formula 0% represents no difference between the amplitude of startle triggered only by the pulse or pulse preceded by the pre-pulse and therefore no pre-pulse inhibition. This transformation was performed in order to reduce the statistical variability attributable to differences between animals and represents a direct measure of pre-pulse inhibition (Issy et al., 2009).

### Statistical Analysis

The percentage of PPI was analyzed by repeated measures MANOVA with the treatment as the independent factor and the prepulse intensity (80, 85 and 90 dB) as repeated measure. Duncan's post hoc test (P<0.05) was used to identify differences revealed by significant MANOVA.

### Results

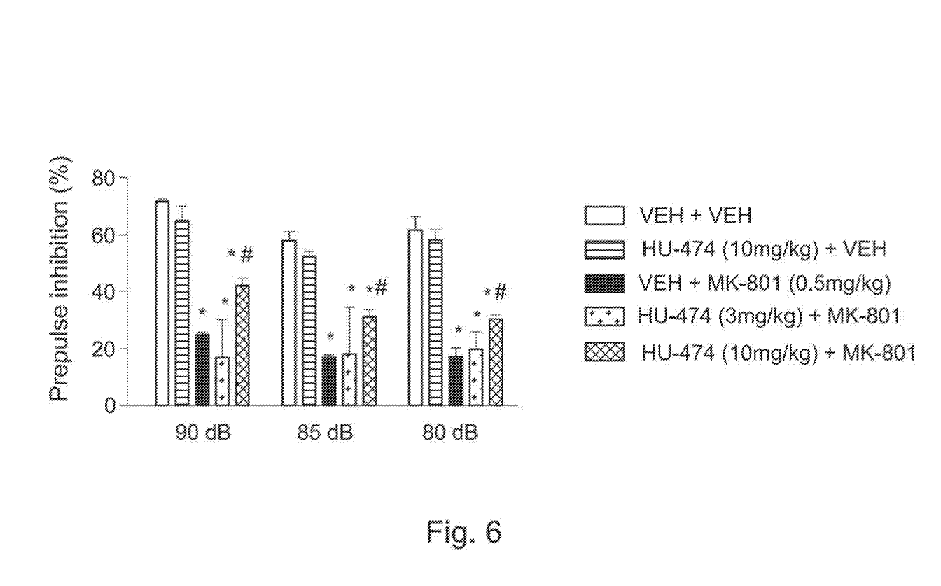

The MANOVA revealed significant main effects of prepulse intensity (F2,70=23.53, P<0.05) and treatment (F4,35=45.42, P<0.05) but no interaction between prepulse intensity and treatment (F8,70=1.08, P>0.05). MK-801 promoted significant PPI disruption for all prepulse intensities tested (P<0.05, Duncan post-test). HU-474 (10 mg/kg) attenuated MK-801 PPI disruption in all prepulse intensities tested (P<0.05, FIG. 6). FIG. 6 shows the effects of HU-474 (3 and 10 mg/kg i.p.) in mice on the impairment of PPI induced by MK-801 (M0.5 mg/kg). Results show the percent inhibition of startle amplitude in response to multiple presentations of the pulse preceded by pre-pulse and are expressed as means±SEM. * indicates significant difference from vehicle-vehicle, # significant difference from vehicle-MK group.

Discussion (Comparison with CBD Results)

A single CBD administration (5 mg/kg, I.p.) attenuated PPI deficits caused by MK801 (0.3-1 mg/kg, i.p.) in Swiss mice (Long et al., 2006). Observe that in this case the effective CBD dose (the authors also tested 1 and 15 mg/kg) was lower than that observed in HU-474 (10 mg/kg).

The above experimental procedure is also performed with 30 mg/kg dose of HU-474. HU-474 is also tested in dopamine-based models (hyperlocomotion induced by d-amphetamine). CBD effective doses are 30 and 60 mg/kg (Swiss mice). 30 mg/kg dose are able to attenuate the hyper-locomotion induced by MK801 (Moreira and Guimarães, 2005).

### Example 9: Effects of HU-485 in Animal Models Predictive of Anxiolytic, Antidepressant and Antipsychotic Effects

### Methods

### 1. Animals

Male Swiss mice (25-30 g) originated from the Central Animal Farm of the School of Medicine of Ribeirão Preto (FMRP-USP) were maintained in groups of five animals per box (41×33×17 cm) in a temperature controlled room (24±2° C.) with a 12×12 h light-dark cycle. They received water and food ad libitum throughout the study period.

### 2. Drug Compositions

Cannabidiol (CBD; THC Pharm, 15-60 mg/kg) and HU-485 (1-10 mg/kg) were administered intraperitoneally (ip). All drugs were dissolved in 2% Tween 80 in sterile saline.

### 3. Behavioral Tests

### 3.1 Elevated Plus-Maze (EPM)

The wood-made EPM was located in a sound attenuated and temperature controlled room (23° C.), with one incandescent light (40 W) placed 1.3 m away from the maze. The apparatus consisted of two opposing open arms (30×5 cm) perpendicular to two enclosed arms (30×5×40 cm), with a central platform common to all arms (5×5 cm). The apparatus was elevated 50 cm above the ground and an acrylic edge (1 cm) surrounded the open arms to prevent animal falls. In this model, anxiolytic drugs typically increase the exploration of the open arms without affecting the number of enclosed arms entries, which is usually used as a measure of general exploratory activity (File, 1992). Twenty minutes after the injections the animals were placed on the central platform of the maze facing one of the enclosed arms. The test lasted for 5 min and the animal behavior was analyzed with the help of the Anymaze Software (version 4.5, Stoelting), which indicated the position of the animal in the maze and calculated the percentage of entries (Peo) and time spent in the open arms (Pto) and the number of entries in the enclosed arms (EA). Animals were only considered to enter an open or enclosed arm when 90% of their bodies were inside the region. All experiments were performed in the morning period (8 to 12 a.m.).

### 3.2 Forced Swimming Test (FST)

Animals were submitted to 6 min of forced swimming in glass cylinders (height 25 cm, diameter 17 cm) containing 10 cm of water at 23-25° C. Immobility time (characterized by slow movements necessary to avoid drowning) was measured during the last 4-min period. The water was changed after each trial to prevent the influence of alarm substances (Zanelati et al., 2010). Antidepressants typically decrease immobility time in this test.

### 3.3 Pre-Pulse Inhibition (PPI)

The PPI test was conducted simultaneously in two identical startle response systems (Med Associates, USA). A continuous acoustic signal provided a background white noise level of 65 dB. The pulse consists of a 105 dB white noise burst with a rise/decay of 5 ms and duration of 20 ms. The pre-pulse comprised pure 7000 Hz tones, 10 ms duration, with intensities set at 80, 85, and 90 dB. The setups were daily calibrated to ensure equal sensitivity throughout the experiments. Calibration was performed by adjusting the gain on the load cell amplifier to 150 arbitrary units (AU) at a standard weight appropriated for a 40 g mice. The limits of the load cell were −2047 to +2047 AU. Thirty min after the injection of the tested compounds mice received i.p. injections of amphetamine 10 mg/kg or vehicle. After a 5 min acclimatization period in which the animal did not listen to any stimuli except the 65 dB background noise, mice were presented with a series of 10 stimuli (pulse alone). The first 10 pulse-alone trials allow for the within-session habituation to the startle stimulus and are not considered for PPI statistical analysis. The test consisted of 64 pseudo-random trials divided into eight different groups presented with an inter-stimulus interval of 30 s, and consisting of pulse alone (105 dB), pre-pulse alone (80, 85, or 90 dB), pre-pulse+pulse with 100 ms interval between pre-pulse and pulse, and no stimulus presented. Pre-pulse stimulus did not elicit an acoustic startle response. Mean acoustic startle response to pulse-alone (P) trials and each pre-pulse+pulse (PP+P) trial was recorded for each subject. PPI was calculated by expressing the pre-pulse+pulse startle amplitude as a percentage of decrease from pulse-alone startle amplitude, according to the following formula: % PPI=100−[100×(PP+P/P)]. This transformation reduces statistical variability attributable to differences between animals and it is a direct PPI measure. Antipsychotic drugs typically attenuate PPI impairment induced by amphetamine.

### 4. Statistical Analysis

Results were analyzed by one-way ANOVA followed by Duncan test. Significant level was set at p<0.05.

### Results

### 1. Elevated Plus Maze and Forced Swimming Tests

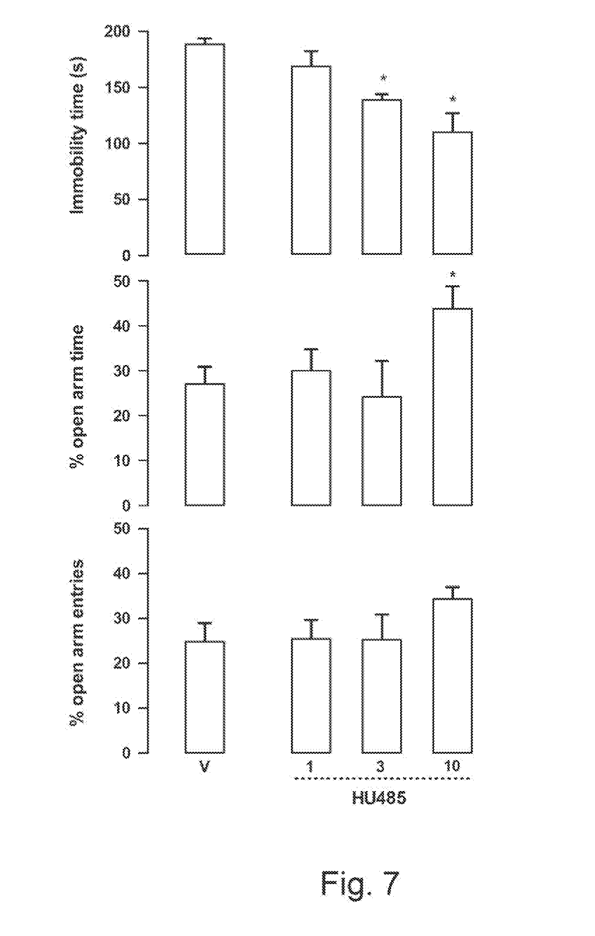

HU-485: At the doses of 3 and 10 mg/kg HU-485 decreased immobility time (F4,22=5.35, p=0.004, Duncan, p<0.05, FIG. 7). The later dose also increased the percentage of time spent in the open arms of the EPM (F3,19=3.08, p=0.052, Duncan, p<0.05, FIG. 7) without changing the number of enclosed arm entries.

FIG. 7 shows the effect of HU-485 (1, 3 and 10 mg/kg, n=5, 5 and 6 animals, respectively) in Swiss male mice tested in the forced swim test (FST, upper panel) and elevated plus maze (EPM). Animals received an injection of vehicle (V, n=7 animals/group) or HU-485 and were tested in the EPM 20 min later. Immediately after the test they were submitted to the FST for 6 min. Data represents the means±SEM of immobility time in the FST and the % of open arm entries and time spent in these arms of the EPM. * indicates difference from V group.

### 2. PPI Results

### CBD:

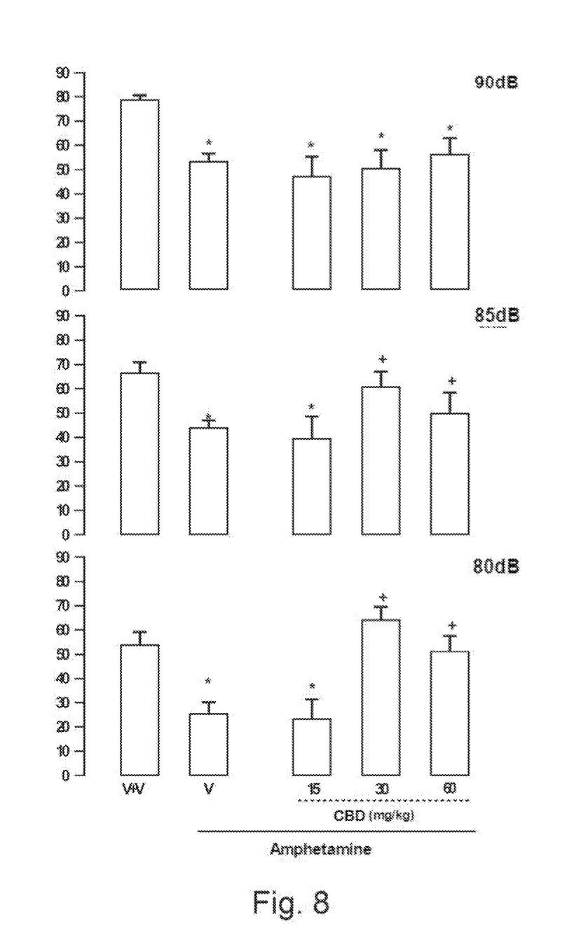

There were significant effects of intensity (F2,70=23.7, p<0.001); treatment (F4,35=5.94, p=0.001) and interaction between factors (F8,70=4.45, p<0.001). Posthoc analysis showed that amphetamine impaired PPI in all intensities (Duncan, p<0.05). This effect was attenuated by CBD at 30 and 60 mg/kg at the intensities of 85 and 80 dB (FIG. 8).

FIG. 8 shows the effect of CBD (15, 30 and 60 mg/kg, n=10 animals/group) in Swiss male mice tested in the pre-pulse inhibition model. Animals received a first injection of vehicle (V) or CBD, followed 30 min later, by vehicle (n=9) or amphetamine 10 mg/kg (V+amphetamine group=9 animals). PPI was evaluated at three-stimuli intensity (90, 85 and 80 dB). Data represents the means±SEM. * indicates difference from V+V group. + indicates difference from V+amphetamine group (p<0.05).

### HU-485:

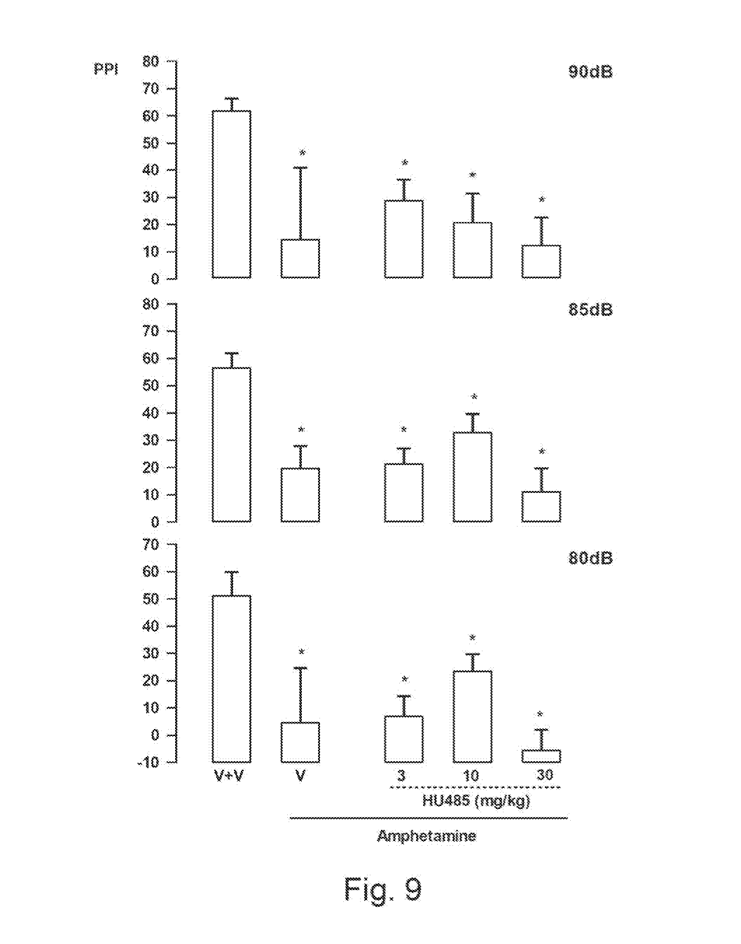

There were significant effects of intensity (F2,72=6.67, p=0.002) and treatment (F5,36=8.53, p<0.001) but no interaction between factors (F10,72=0.80, NS). Posthoc analysis showed that amphetamine impaired PPI in all intensities (Duncan, p<0.05). This effect was not attenuated by HU-485 at any dose (FIG. 9). By itself HU-485 30 mg/kg did not impair PPI.

FIG. 9 shows the effect of HU-485 (3, 10 and 30 mg/kg, n=7-8 animals/group) in Swiss male mice tested in the pre-pulse inhibition model. Animals received a first injection of vehicle (V) or HU-485, followed 30 min later, by vehicle (n=7) or amphetamine 10 mg/kg (V+amphetamine group=7 animals). PPI was evaluated at three-stimuli intensity (90, 85 and 80 dB). Data represents the means±SEM. * indicates difference from V+V group (p<0.05).

### Example 10: Cannabinoid Receptor CB2 Binding for HU-487

For CB1 receptor binding assay synaptosomal membranes from rat brains were used. Sabra male rats weighing ˜200 g were decapitated and their brains, without the brain stem, were quickly removed. Synaptosomal membranes were prepared from the brains by centrifugation and sucrose density gradient ultracentrifugation after their homogenization. The CB2 receptor binding assays were performed using crude membranes obtained from Chinese hamster ovary (CHO) cells stably transfected with the human CB2 cDNA.

The high affinity CB1/CB2 receptor ligand, [3H]CP-55,940, with a dissociation constant of 2 nM for CB1 and CB2, respectively, was incubated for 90 min at 30° C. with synaptosomal membranes for CB1, or with transfected cells for CB2 in the presence of the tested CBD derivatives. Bound and free radioligand were separated by centrifugation (13,000 rpm for 6 min). Non-specific binding, which was determined with 50 nM unlabelled CP-55,940, was subtracted. Binding experiments were repeated 2-3 times and each point performed in triplicate. The Ki values were determined using the GraphPad Prism program (La Jolla, Calif., USA) and the Cheng-Prusoff equation.

CB2(Ki) for HU-487 was determined to be 4-5 μM.

### REFERENCES

Campos A C, Guimarães F S. Involvement of 5HT1A receptors in the anxiolytic-like effects of cannabidiol injected into the dorsolateral periaqueductal gray of rats. Psychopharmacology (Burl). 199(2):223-30, 2008.
File S E. Behavioural detection of anxiolytic action, in Experimental approaches to anxiety and depression (Elliott J M, Heal D J and Marsden C A eds) pp 25-44, Wiley, New York, 1992.
Issy A C, Salum C, Del Bel E A. Nitric oxide modulation of methylphenidate-induced disruption of prepulse inhibition in Swiss mice. Behav Brain Res. 205(2):475-81, 2009.
Long L E, Malone D T, Taylor D A. Cannabidiol Reverses MK-801-Induced Disruption of Prepulse Inhibition in Mice. Neuropsychopharmacology 31:795-803, 2006.
Moreira F A, Guimarães F S. Cannabidiol inhibits the hyperlocomotion induced by psychotomimetic drugs in mice. Eur J Pharmacol. 512(2-3):199-205, 2005.
Onaivi E S, Green M R, Martin B R. Pharmacological characterization of cannabinoids in the levated plus maze J Pharmacol Exp Ther 255:1002-9, 1990.
Paxinos G. and Watson C. The rat brain in stereotaxic coordinates. Academic Press, New York, 1997.
Zanelati T V, Biojone C, Moreira F A, Guimarães F S, Joca S R. Antidepressant-like effects of cannabidiol in mice: possible involvement of 5-HT1A receptors. Br J Pharmacol. 159:122-8, 2010.

In addition, related disclosure as described in commonly-owned International Publication No. WO2014/108899 is herein incorporated by reference in its entirety for all purposes.

Acknowledgement of the above references herein is not to be inferred as meaning that these are in any way relevant to the patentability of the presently disclosed subject matter. Each cited application and journal article is incorporated by reference in its entirety for all purposes.

1. A compound having the general formula (I): 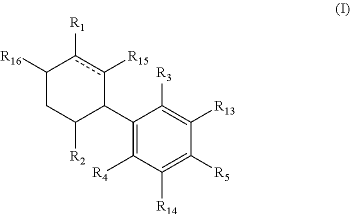
wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R2 is selected from straight or branched C1-C8 alkyl, straight or branched C2-C10 alkenyl, straight or branched C2-C10 alkynyl, each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is selected from a straight or branched C5-C12 alkyl, a straight or branched C5-C9 alkoxy, a straight or branched C1-C7 ether, each being optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13, R14, R15 and R16 are each optionally selected from H and F;
provided that at least one of R13, R14, R15 and R16 is F or at least one of R1 and R2 is substituted with F.

2. A compound having the general formula (Ia): 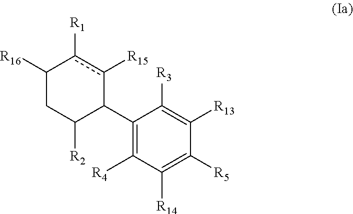
wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R2 is selected from straight or branched C1-C8 alkyl, straight or branched C2-C10 alkenyl, straight or branched C2-C10 alkynyl, each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is a straight or branched C5 alkyl, optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13, R14, R15 and R16 are each optionally selected from H and F;
provided that at least one of R13, R14, R15 and R16 is F or at least one of R1 and R2 is substituted with F.

3. A compound having the general formula (II): 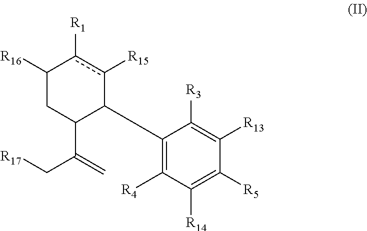
wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is selected from a straight or branched C5-C12 alkyl, a straight or branched C5-C9 alkoxy, a straight or branched C1-C7 ether, each being optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13, R14, R15, R16 and R17 are each optionally selected from H and F;
provided that at least one of R13, R14, R15 and R16 is F or R1 is substituted with F.

4. A compound having the general formula (IIa): 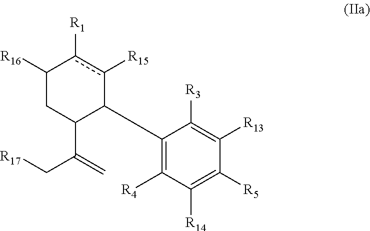
wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is a straight or branched C5 alkyl, optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13, R14, R15, R16 and R17 are each optionally selected from H and F;
provided that at least one of R13, R14, R15 and R16 is F or R1 is substituted with F.

5. A compound having the general formula (III): 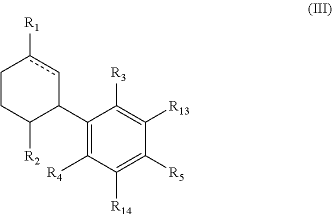
wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9;
R2 is selected from straight or branched C1-C8 alkyl, straight or branched C2-C10 alkenyl, straight or branched C2-C10 alkynyl;
R3 and R4 are each independently selected from H, straight or branched C1-C5 alkyl, —OR10, —C(═O)R11, —OC(═O)R12; provided that at least one of R3 and R4 is different than H;
R5 is selected from a straight or branched C5-C12 alkyl, a straight or branched C5-C9 alkoxy, a straight or branched C1-C7 ether, each being optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13 and R14 are each optionally selected from H and F;
provided that at least one of R13 and R14 are F.

6. A compound having the general formula (IIIa): 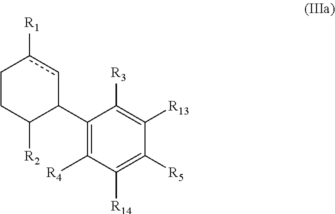
wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9;
R2 is selected from straight or branched C1-C8 alkyl, straight or branched C2-C10 alkenyl, straight or branched C2-C10 alkynyl;
R3 and R4 are each independently selected from H, straight or branched C1-C5 alkyl, —OR10, —C(═O)R11, —OC(═O)R12; provided that at least one of R3 and R4 is different than H;
R5 is a straight or branched C5 alkyl, optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R13 and R14 are each optionally selected from H and F;
provided that at least one of R13 and R14 are F.

7. A compound of general formula (IV) 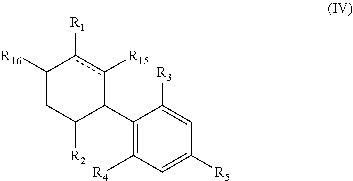
wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R2 is selected from straight or branched C1-C8 alkyl, straight or branched C2-C10 alkenyl, straight or branched C2-C10 alkynyl, each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is selected from a straight or branched C5-C12 alkyl, a straight or branched C5-C9 alkoxy, a straight or branched C1-C7 ether, each being optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R15 and R16 are each optionally selected from H and F;
provided that at least one of R15 and R16 is F or at least one of R1 and R2 is substituted with F.

8. A compound of general formula (IVa) 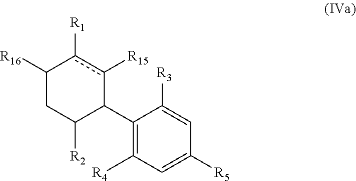
wherein [symbol]
is a single or double bond;
R1 is selected from straight or branched C1-C8 alkyl, straight or branched C2-C10 alkenyl, straight or branched C2-C10 alkynyl, —C(═O)R8, —C(═O)OR9 each optionally substituted by at least one F;
R2 is selected from straight or branched C1-C8 alkyl, straight or branched C2-C10 alkenyl, straight or branched C2-C10 alkynyl, each optionally substituted by at least one F;
R3 and R4 are each independently selected from H, straight or branched C1-C5 alkyl, —OR10, —C(═O)R11, —OC(═O)R12; provided that at least one of R3 and R4 is different than H;
R5 is a straight or branched C5 alkyl, optionally substituted by at least one substituent selected from —OH, —NH3, straight or branched C1-C5 amine, halogen, phenyl, aryl, heteroaryl, cycloalkyl and heterocycloalkyl;
R8, and R9 are independently selected from H, OH, straight or branched C1-C5 alkyl, straight or branched C1-C5alkoxy, —NH3, straight or branched C1-C5 amine;
R10 is selected from H, a straight or branched C1-C5 alkyl; and
R11 and R12 are independently selected from H, OH, straight or branched C1-C5 alkyl, straight or branched C1-C5 alkoxy, —NH3, straight or branched C1-C5 amine;
R15 and R16 are each optionally selected from H and F;
provided that at least one of R15 and R16 is F or at least one of R1 and R2 is substituted with F.

9. The compound of claim 1 or 2, wherein

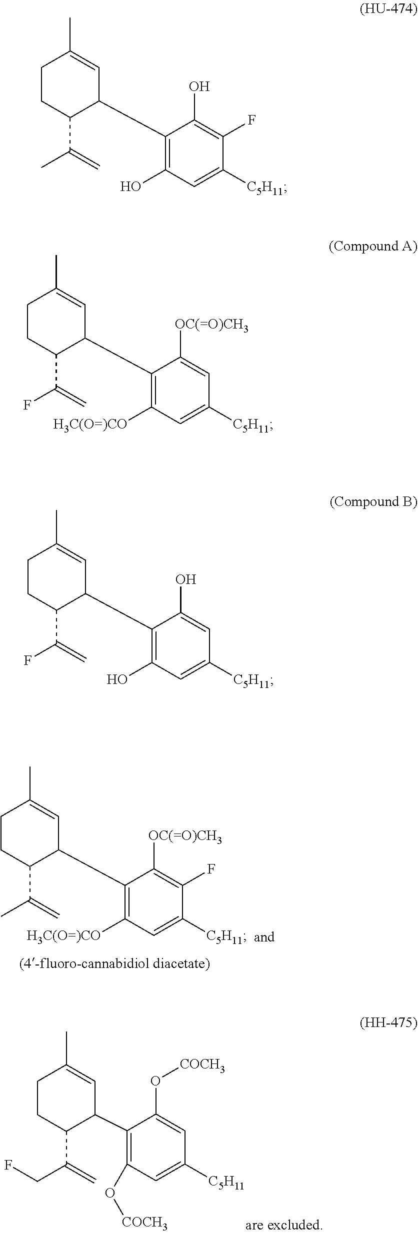

10. The compound according to any of claims 3-6, wherein HU-474 and 4′-fluoro cannabidiol diacetate are excluded.

11. The compound of claim 7 or 8, wherein HU-475, Compound A, and Compound B are excluded.

12. A compound according to any one of the preceding claims, wherein [symbol] is a double bond.

13. A compound according to any one of preceding claims, wherein R1 straight or branched C1-C8 alkyl; R3 and R4 are each independently —OR10; R10 is selected from H, a straight or branched C1-C5 alkyl.

14. A compound according to any one of preceding claims, wherein R1 is straight or branched C1-C8 alkyl, and R3 and R4 are OH.

15. A compound according to any one of the preceding claims, wherein R3 and R4 are each independently selected from H, —OR10, and —OC(═O)R12; R10 is selected from H, a straight or branched C1-C5 alkyl; and R12 is selected from H, OH, straight or branched C1-C5 alkyl, —NH3, straight or branched C1-C5 amine.

16. A compound according to any one of claims 1, 3, 5, 7, or 12-15, wherein R5 is a straight or branched C5-C12 alkyl.

17. A compound according to any one of claims 1, 3, 5, 7, or 12-16, wherein R5 is a straight or branched C5 alkyl.

18. A compound according to claim 1, wherein at least one of R13, R14, R15 and R16 is F.

19. A compound according to claim 1, wherein at least one of R13 and R14 is F.

20. A compound according to claim 1, wherein at least one of R15 and R16 is F.

21. A compound according to claim 1, wherein at least one of R1 and R2 is substituted with F.

22. A compound according to claim 13, wherein R1 is selected from straight or branched C1-C8 alkyl, straight or branched C2-C10 alkenyl, straight or branched C2-C10 alkynyl, each substituted by F.

23. A compound according to claim 13, wherein R2 is selected from straight or branched C1-C8 alkyl, straight or branched C2-C10 alkenyl, straight or branched C2-C10 alkynyl, each substituted by F.

24. A compound according to any one of the preceding claims, having the general formula (V): 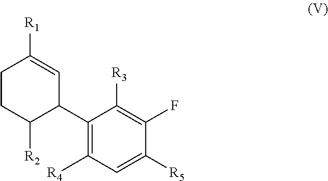
wherein R1, R2, R3, R4 and R5 are as defined therein.

25. The compound of claim 24, wherein R5 is a straight or branched C5-C12 alkyl.

26. The compound of claim 24, wherein R5 is a straight or branched C5 alkyl

27. The compound of claim 25 or 26, wherein HU-474 and 4′-fluoro cannabidiol diacetate are excluded.

28. A compound according to any one of the preceding claims, having the general formula (VI): 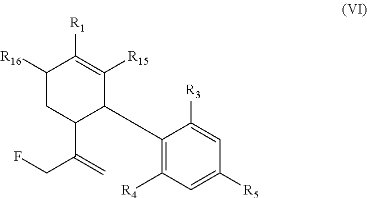
wherein R1, R3, R4, R5, R15 and R16 are as defined therein.

29. The compound of claim 27, wherein R5 is a straight or branched C5-C12 alkyl.

30. The compound of claim 27, wherein R5 is a straight or branched C5 alkyl.

31. The compound of claim 31, wherein HU-475 is excluded.

32. A compound selected from one of the following:

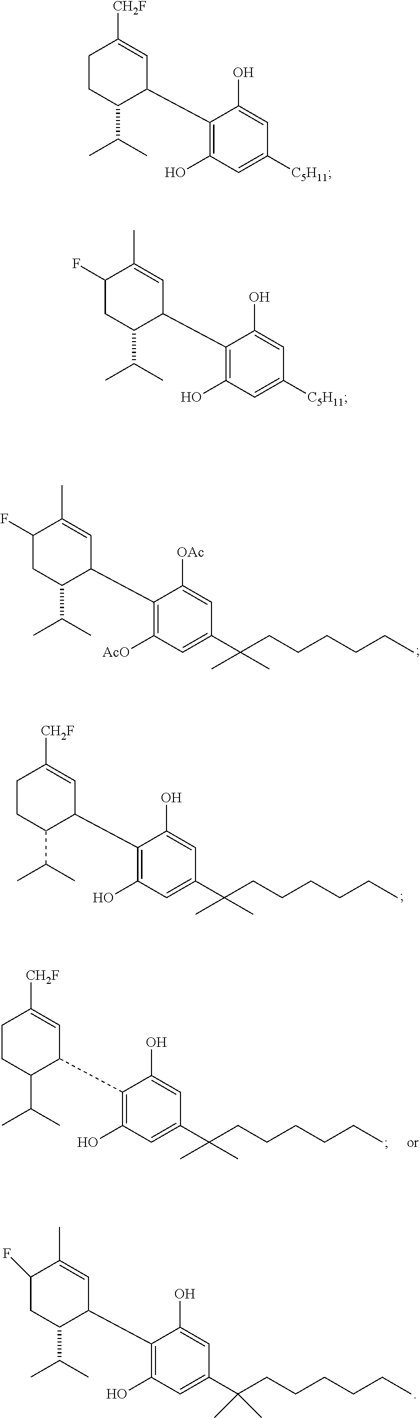

33. A composition comprising at least one compound according to any one of claims 1 to 32.

34. A composition according to claim 33, wherein said composition is a pharmaceutical composition.

35. A composition comprising at least one compound according to any one of claims 1 to 32, being an antioxidant composition.

36. A compound according to any one of claims 1 to 32, for use in the treatment of at least one condition, disease or disorder selected from the group consisting of psychiatric disorders, inflammation, oxidation associated conditions, rheumatoid arthritis, cardiovascular diseases, obesity, diabetes and associated disorders and symptoms, emesis and nausea, ischemic/reperfusion injury associated with myocardial, liver or renal diseases, hypoxia/ischemia injury, neuronal damage due to neurological diseases or injury, cancer and resistance to cancer chemotherapy, epilepsy and convulsions, and any condition or symptom associated therefrom.

37. A compound according to claim 36, wherein said condition, disease, disorder or symptom associated with inflammation is selected from rheumatoid arthritis, multiple sclerosis, inflammatory bowel disease, diabetes and any combinations thereof.

38. A compound according to claim 36, wherein said disease is a psychiatric disease condition or disorder or any symptom associated therewith.

39. A compound according to claim 38, wherein said psychiatric disease condition or disorder or any symptom associated therewith is selected from anxiety, stress, depression, schizophrenia, panic, substance abuse withdrawal symptoms, reward-facilitating effect of addictive substances, memory loss, psychotic-like symptoms associated with the use of substance abuse.

40. A compound according to any one of claims 1 to 32, for use in reduction of oxidative stress.

41. A compound according to any one of claims 1 to 32, for use in the treatment of any disease, condition or disorder caused by or associated with oxidative stress.

42. A compound according to any claim 41, wherein said disease, condition or disorder caused or associated with oxidative stress are selected from the group consisting of cancer, oxidative neurological disorders, free radical associated diseases, ischemia, ischemic reperfusion injury, inflammatory diseases, systemic lupus erythematosis, myocardial ischemia or infarction, cerebrovascular accidents, operative ischemia, traumatic hemorrhage, spinal cord trauma, Down's syndrome, Crohn's disease, autoimmune diseases, cataract formation, uveitis, emphysema, gastric ulcers, oxygen toxicity, neoplasia, undesired cellular apoptosis, radiation sickness, and any combinations thereof.

43. A compound according to any one of claims 1 to 32, for use in the treatment of oxidative associated diseases, disorder or condition of the CNS.

44. A compound according to any one of claims 1 to 32, for use in preventing, arresting, or treating neurological damage in a subject suffering from at least one disease, disorder or condition selected from Parkinson's disease, Alzheimer's disease and HIV dementia; autoimmune neurodegeneration, hypoxic or anoxic neuronal damage, respiratory arrest or cardiac arrest, anoxia caused by drowning and brain surgery or trauma or any combinations thereof.

45. A compound according to any one of claims 1 to 32, for use in the treatment of an ischemic or neurodegenerative disease in the central nervous.

46. A compound according to claim 45, wherein the ischemic or neurodegenerative disease is selected from the group consisting of: an ischemic infarct, Alzheimer's disease, Parkinson's disease, and human immunodeficiency virus dementia, Down's syndrome, and heart disease or any combinations thereof.

47. Use of a compound according to any one of claims 1 to 32, for the manufacture of a medicament.

48. Use of a compound according to any one of claims 1 to 32, for the manufacture of a medicament for the treatment of at least one condition, disease or disorder selected from the group consisting of psychiatric disorders, inflammation, oxidation associated conditions, rheumatoid arthritis, cardiovascular diseases, obesity, diabetes and associated disorders and symptoms, emesis and nausea, ischemic/reperfusion injury associated with myocardial, liver or renal diseases, hypoxia/ischemia injury, neuronal damage due to neurological diseases or injury, cancer and resistance to cancer chemotherapy, epilepsy and convulsions, and any condition or symptom associated therefrom.

49. Use of a compound according to any one of claims 1 to 32, for the manufacture of a medicament for reduction of oxidative stress.

50. Use of a compound according to any one of claims 1 to 32, for the manufacture of a medicament for the treatment of any disease, condition or disorder caused or associated with oxidative stress.

51. Use according to claim 50, wherein said disease, condition or disorder caused or associated with oxidative stress are selected from the group consisting of cancer, oxidative neurological disorders, free radical associated diseases, ischemia, ischemic reperfusion injury, inflammatory diseases, systemic lupus erythematosis, myocardial ischemia or infarction, cerebrovascular accidents, operative ischemia, traumatic hemorrhage, spinal cord trauma, Down's syndrome, Crohn's disease, autoimmune diseases, cataract formation, uveitis, emphysema, gastric ulcers, oxygen toxicity, neoplasia, undesired cellular apoptosis, radiation sickness, and any combinations thereof.

52. Use of a compound according to any one of claims 1 to 32, for the manufacture of a medicament for the treatment of oxidative associated disease, disorder or condition of the CNS.

53. Use of a compound according to any one of claims 1 to 32, for the manufacture of a medicament for preventing, arresting, or treating neurological damage in a subject suffering from at least one disease, disorder or condition selected from Parkinson's disease, Alzheimer's disease and HIV dementia; autoimmune neurodegeneration, hypoxic or anoxic neuronal damage, respiratory arrest or cardiac arrest, anoxia caused by drowning and brain surgery or trauma and any combinations thereof.

54. Use of a compound according to any one of claims 1 to 32, for the manufacture of a medicament for the treatment of an ischemic or neurodegenerative disease in the central nervous.

55. Use according to claim 54, wherein the ischemic or neurodegenerative disease is selected from the group consisting of: an ischemic infarct, Alzheimer's disease, Parkinson's disease, and human immunodeficiency virus dementia, Down's syndrome, and heart disease or any combinations thereof.

56. A method of treating a condition, disease, disorder or symptom associated with inflammation in a subject in need thereof, said method comprising administering to said subject an effective amount of at least one compound according to any one of claims 1 to 32.

57. A method of reduction of oxidative stress in a tissue or an organ of a subject in need thereof, said method comprising administering to said subject an effective amount of at least one compound according to any one of claims 1 to 32.

58. A method of treating any disease, condition or disorder caused by or associated with oxidative stress a subject in need thereof, said method comprising administering to said subject an effective amount of at least one compound according to any one of claims 1 to 32.

59. A method according to claim 58, wherein said disease, condition or disorder caused by or associated with oxidative stress are selected from the group consisting of cancer, oxidative neurological disorders, free radical associated diseases, ischemia, ischemic reperfusion injury, inflammatory diseases, systemic lupus erythematosis, myocardial ischemia or infarction, cerebrovascular accidents, operative ischemia, traumatic hemorrhage, spinal cord trauma, Down's syndrome, Crohn's disease, autoimmune diseases, cataract formation, uveitis, emphysema, gastric ulcers, oxygen toxicity, neoplasia, undesired cellular apoptosis, radiation sickness, and any combinations thereof.

60. A method for the treatment of oxidative associated disease, disorder or condition of the CNS in a subject, comprising administering to said subject a therapeutically effective amount of a compound of any one of claims 1 to 32.

61. A method for preventing, arresting, or treating neurological damage in a subject suffering from at least one disease, disorder or condition selected from Parkinson's disease, Alzheimer's disease and HIV dementia; autoimmune neurodegeneration, hypoxic or anoxic neuronal damage, respiratory arrest or cardiac arrest, anoxia caused by drowning and brain surgery or trauma and any combinations thereof, comprising administering to said subject a therapeutically effective amount of a compound of any one of claims 1 to 32.

62. A method of treating an ischemic or neurodegenerative disease in the central nervous system of a subject, comprising administering to the subject a therapeutically effective amount of a compound according to claims 1 to 32.

63. A method according to claim 62, wherein the ischemic or neurodegenerative disease is selected from the group consisting of: an ischemic infarct, Alzheimer's disease, Parkinson's disease, and human immunodeficiency virus dementia, Down's syndrome, and heart disease or any combinations thereof.
